(19) United States
(12) Patent Application Publication
(10) Pub. No.: US 2019/0038588 A1
(43) Pub. Date: Feb. 7, 2019

(54) NOVEL CLASS OF COMPOUNDS FOR THE TREATMENT OF CARDIOVASCULAR DISEASE

(71) Applicant: STICHTING KATHOLIEKE UNIVERSITEIT, Nijmegen (NL)

(72) Inventors: Tom Johan Joseph Schirris, Nijmegen (NL); Tina Ritschel, Oegstgeest (NL); Floris Petrus Johannes Theodorus Rutjes, Wijchen (NL); Johannes Albertus Maria Smeitink, Beuningen (NL); François Gérard Marie Russel, Nijmegen (NL)

(73) Assignee: STICHTING KATHOLIEKE UNIVERSITEIT, Nijmegen (NL)

(21) Appl. No.: 16/075,200

(22) PCT Filed: Feb. 9, 2017
(86) PCT No.: PCT/EP2017/052807

§ 371 Date: Aug. 3, 2018

(30) Foreign Application Priority Data
Feb. 11, 2016 (EP) 16155245.0

(51) Int. Cl. A61K 31/215; A61K 31/404; A61P 25/28; A61K 31/505; A61K 31/426; A61K 31/421; A61K 31/4164; A61P 9/10; A61K 31/4025; A61K 31/381; A61K 31/365; A61P 3/06; A61K 45/06

(52) U.S. Cl. CPC A61K 31/215; A61K 31/404; A61P 25/28; A61K 31/505; A61K 31/426; A61K 31/421; A61K 2300/00; A61P 9/10; A61K 31/4025; A61K 31/381; A61K 31/365; A61P 3/06; A61K 45/06; A61K 31/4164

(57) ABSTRACT

The present invention relates to the field of medicine, specifically the field of treatment and prevention of cardiovascular diseases.

### FIELD OF THE INVENTION

The present invention relates to the field of medicine, specifically the field of treatment and prevention of cardiovascular diseases.

### BACKGROUND OF THE INVENTION

Cardiovascular diseases are still one the most frequent causes of mortality. Multiple risk factors have been identified. Reduction of these risk factors has significantly reduced the mortality rates due to cardiovascular diseases. High blood cholesterol levels, and especially high levels of the low-density lipoproteins (LDL), have been indicated as an important risk factor. Over the past decades multiple drug treatments have been developed to lower these blood cholesterol levels, such as statins, niacin, niacin derivatives such as its eicosapentaenoic acid conjugate (ARI-30337MO), bile-acid resins, fibric acid derivatives, and cholesterol absorption inhibitors (e.g. ezetimibe). More recent efforts to obtain good treatment methods have focused on proprotein convertase subtilisin/kexin type 9 (PCSK9) inhibitors, cholesteryl ester transfer protein (CEPT) inhibitors, bempedoic acid (a dual adenosine triphosphate citrate lyase inhibitor/adenosine monophosphate-activated protein kinase activator), apolipoprotein B (Apo B) inhibitors, peroxisome proliferator-activated receptor (PPAR) delta agonists, acetyl coenzyme A carboxylase inhibitors, and angiopoietin-like 3 (ANGPTL3) inhibitors (Turner and Stein 2015; Sahebkar and Watts 2013). Of these treatment options, statins seem to be most effective in lowering the risk on major cardiovascular events to date. 3-Hydroxy-3-methyl-glutaryl-coenzyme A reductase (sometimes abbreviated as HMG-CoA reductase, or HMGCR, or HMGR) (Haines et al, 2013) is the rate-controlling enzyme (NADH-dependent, EC 1.1.1.88; NADPH-dependent, EC 1.1.1.34) of the mevalonate pathway, the metabolic pathway that produces cholesterol and other isoprenoids. 3-hydroxy-3-methyl-glutaryl moiety is often referred to as hydroxymethylglutaryl, or abbreviated to HMG. Coenzyme A is routinely abbreviated CoA. HMG-CoA is 3-hydroxy-3-methylglutaryl-coenzyme A, an intermediate in the mevalonate and ketogenesis pathways. Herein, this catalytic entity will generally be referred to as HMG-CoA reductase.

Mitochondrial complex III is also known as ‘coenzyme Q:cytochrome c—oxidoreductase’, or as ‘the cytochrome bc₁ complex’, and is sometimes referred to as Complex III (Dröse and Brandt, 2012). It is the third complex in the electron transport chain (EC 1.10.2.2), playing a critical role in biochemical generation of ATP (oxidative phosphorylation). Complex III is a multisubunit transmembrane protein encoded by both the mitochondrial (cytochrome b) and the nuclear genomes (all other subunits). In this document, this catalytic entity will generally be referred to as Complex III.

Many statins are known, and commercially available. All share an HMG-like moiety of general formula IV, which is a type of dihydroxypentanoic acid moiety, which may be present in an inactive lactone form of general formula V. In vivo, these lactones (V) can again be hydrolyzed, sometimes enzymatically, to their active carboxylic acid forms (IV). Active carboxylic acid forms (IV) can in turn be converted to inactive lactones (V) by uridine 5′-diphospho-glucuronosyltransferases (UGTs) (Prueksaritanont et al., 2002).

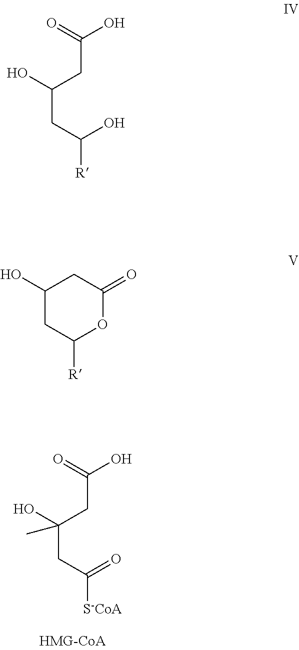

Statins generally share rigid, hydrophobic groups that are covalently linked to the HMG-like moiety of general formulae IV or V. These groups are present at the R′ position depicted in general formulae IV and V. Lovastatin (also known as mevacor, altocor, or altoprev), pravastatin (also known as pravachol, selektine, or lipostat), and simvastatin (also known as zocor or lipex) resemble the substituted decalin-ring structure of mevastatin (also known as compactin). Fluvastatin (also known as lescol, or lescol XL), cerivastatin (also known as lipobay, or baycol), atorvastatin (also known as lipitor, or torvast), pitavastatin (also known as livalo, or pitava), and rosuvastatin (also known as crestor) are fully synthetic HMG-CoA reductase inhibitors with different groups linked to the HMG-like moiety of general formula IV. These different groups range in character from very hydrophobic (e.g., cerivastatin) to partly hydrophobic (e.g., rosuvastatin).

All statins are competitive inhibitors of HMG-CoA reductase with respect to binding of the substrate HMG-CoA, but not with respect to binding of NADPH (Endo et al., 1976). The IC₅₀ values for statins are in the range between 5 and 46 nM, as follows: pravastatine (46 nM), fluvastatine (28 nM), simvastatine (11 nM), cerivastatin (10 nM), atorvastatine (8 nM), pitavastatine (7 nM), rosuvastatine (5 nM) (Kajinami K, 2003; Istvan ES, 2001). Their Michaelis-Menten constant, Kₘ, for HMG-CoA is about 4 mM (Bischoff et al., 1992).

Statins lower systemic cholesterol levels by inhibition of HMG-CoA reductase, the rate limiting enzyme of the cholesterol biosynthesis pathway in the human liver, leading to a decreased endogenous hepatic cholesterol production. Due to their efficacy in lowering cardiovascular risk factors statins are used by hundreds of millions of patients worldwide. Although they are generally well tolerated, side effects occur, statin-induced myopathies being the most common side effects. Although severe muscle breakdown (rhabdomyolysis) or inflammation is only observed in a small fraction of all users, less severe types of muscle pain have been observed in up to 26 percent of all patients. Consequently, up to 30 million patients worldwide are expected to experience such muscle complaints. These adverse effects do directly impair the quality of life of these patients, as they limit these patients in their daily activities. Moreover, it has been shown that these muscle complaints severely lower the therapy adherence, eventually leading to an increasing number of patients unnecessarily being at risk to develop a cardiovascular event. Both consequences are expected to increase the expenditures on treatment of the side effects and cardiovascular events as well as to increase the load of these diseases on the health care system.

Accordingly, there is an urgent need for compounds for the treatment or prevention of high blood cholesterol levels that have less side effects than statins.

### SUMMARY OF THE INVENTION

In an aspect, the present invention provides a compound that inhibits 3-hydroxy-3-methylglutaryl-coenzyme A (HMG-CoA) reductase, while not inhibiting mitochondrial complex III. Preferred compounds are derived from a compound selected from the group consisting of simvastatin, atorvastatin, cerivastatin, fluvastatin, lovastatin, mevastatin, pitavastatin, pravastatin, and rosuvastatin, wherein the carboxylic acid moiety has been replaced by a substituted or non-substituted moiety that acts as a bioisostere.

In a second aspect, the invention provides a pharmaceutical composition comprising such a compound, further comprising a pharmaceutically acceptable excipient. This composition can be in the form of a tablet, soft or hard capsule, ampoule, solution for injection, emulsion or suspension.

In a third aspect, the compound or composition of the invention is for use as a medicament. In a fourth aspect, use of the compound or composition of the invention in the manufacture of a medicament is provided. Medicaments of this invention can be for the treatment, prevention, or delay of a cardiovascular disease, hypercholesterolemia, hypertriglyceridemia, a metabolic disorder, inflammation, nephropathy, and/or Alzheimer's disease in a subject. Accordingly, a fifth aspect provides a method for the treatment of these conditions using a compound or composition of the invention.

In a sixth aspect, the invention provides a method for identifying statin analogues that exhibit a reduced level of mitochondrial complex III inhibition.

### DETAILED DESCRIPTION OF THE INVENTION

A pivotal role of mitochondrial dysfunction was described to underlie the statin-associated side effects described here above (Marcoff and Thompson, 2007; Sirvent et al, 2008). However, the molecular mechanism remained elusive. Combining in vitro and in silico approaches the inventors of the present invention were able to pinpoint the third complex of the mitochondrial respiratory chain as a novel off-target underlying these statin-induced myopathies. The validity of this off-target was confirmed by a decreased activity of Complex III of the respiratory chain in patients suffering from these side effects, and was observed to correlate with the clinical phenotype of these effects (Schirris et al, 2015). It was found that statins caused the side effect in their lactone form of general formula V, and not in their carboxylic acid form of general formula IV. These two forms can convert into one another in vivo, which means that administration of only one of the two forms does not prevent or avoid the in vivo presence of the other form. The inventors have surprisingly found a novel class of compounds that effectively lowers blood cholesterol levels by inhibiting HMG-CoA reductase while not inhibiting mitochondrial complex III. These novel compounds according to the invention are statin-like structures and therefore provide a novel treatment option with an increased safety profile. They cannot form lactones. Similar to the state of the art statins, the statin-like structures according to the invention provide an easy to use drug therapy for the treatment of cardiovascular disease, along with reduction of the muscle pain side effects that results from the absence of a potential lactone form of general formula V. Owing to these reduced side effects, the statin-like structures according to the invention are expected to provide an improved therapy adherence of cardiovascular disease and consequently reduce the morbidity and mortality associated with cardiovascular events.

The novel statin-like structures according to the invention are HMG-CoA reductase inhibitors and have been realized by bioisosteric replacement of the carboxylic acid moiety that is involved in lactone formation. The statin-like structures according to the invention do not inhibit Complex III, which is associated with statin-induced myopathies as described above.

Bioisosteres are structurally different molecules, moieties, or substructures, that can form comparable intermolecular interactions (Ritschel et al, 2012). In the case of the statin-like structures according to the invention, the carboxylic acid group of the dihydroxyheptanoic acid side chain of general formula IV of the pharmacological active statin acid forms has been replaced by bioisosteres, preventing the formation of the toxic lactone form of general formula V in vivo but preserving the hydrogen bond network between the dihydroheptanoic moiety of general formula IV and HMG-CoA reductase. Effectively, this preserves the desired effect of the original statin, while reducing its side effects.

For adequate selection of statin bioisosteres several selection criteria were defined:

i) fit to the binding site of HMG-CoA reductase (sterically, H-bond interactions);

ii) negative charge of the bioisostere;

iii) non-fit to the Qₒ site of Complex III.

Effective bioisosteres according to the invention are presented later herein. The selection criteria here above illustrate that these bioisosteres can be applied to any statin-like compound of general formula IV, and that any statin that features general formula IV is therefore encompassed by the invention. It should be understood that statins of general formula V, such as simvastatin or lovastatin, are similarly encompassed, because such statins can be transformed into statins of general formula IV, and generally do so in vivo. As such, statins of general formulae IV and V are similarly encompassed. The negative charge recited at ii) relates to a negative charge under physiological conditions, or to negative charge under conditions for binding HMG-CoA reductase or Complex III.

Compound

In a first aspect, the present invention provides for a compound that inhibits 3-hydroxy-3-methylglutaryl-coenzyme A (HMG-CoA) reductase while not inhibiting mitochondrial complex III, wherein said compound has the general formula I:

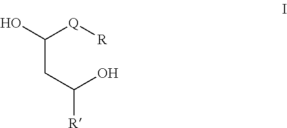

wherein Q is selected from —CH₂— or a bond,
wherein R′ is selected from the group consisting of:

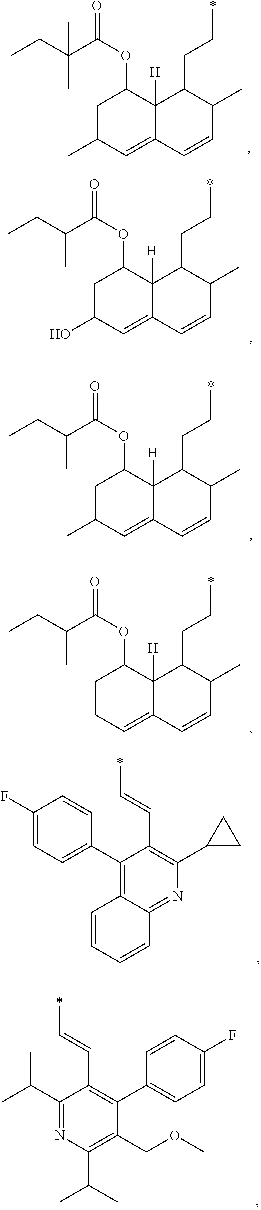

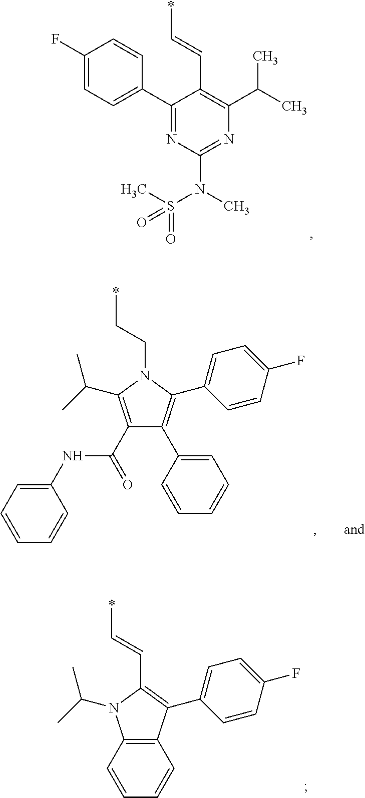

wherein R is a substituted or non-substituted moiety selected from the group consisting of hydroxamic acid, tetrazole, nitrile, nitro, thiocarboxylic acid, amide, sulfonic acid, sulfonamide, phosphonate, boronic acid, 4-linked 3-hydroxycyclobut-3-ene-1,2-dione, an oxo-oxadiazole-like moiety with optional sulfur-substitutions, and moieties having a general formula II or III:

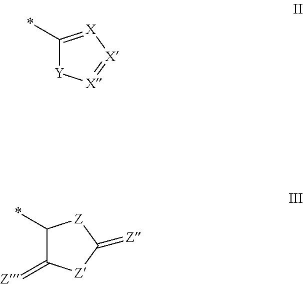

wherein X and X′ and X″ are each individually N, CH, or C—OH,
wherein Y is O, S, NH, or N—CH₃,
wherein Z, Z′, Z″, and Z′″ are each individually O, S, NH, or CH₂, wherein preferably Z″ and Z′″ are each individually O or S,
preferably, wherein R is not carboxylic acid.

Said compound is herein referred to as a compound according to the invention.

Moieties of general formula I are preferably of general formula Ia, Ib, Ic, Id, Ie, If, Ig, or Ih:

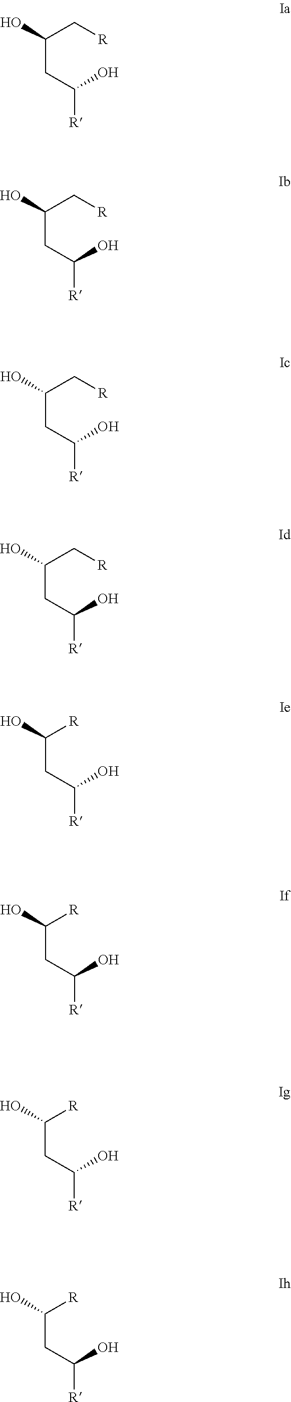

More preferably, these structures are of general formula Ia or of general formula Ie. Most preferably, these structures are of general formula Ia.

As described, Q is selected from —CH₂— or a bond. A bond is a covalent bond, which when Q is a bond directly links R to the rest of general formula I. To illustrate, general formula Ia and general formula Ie differ in the fact that for general formula Ia, Q is —CH₂—, whereas for general formula Ie, Q is a bond. The same difference separates general formula Ib (Q is —CH₂—) from general formula If (Q is a bond), separates general formula Ic (Q is —CH₂—) from general formula Ig (Q is a bond), and separates general formula Id (Q is —CH₂—) from general formula Ih (Q is a bond).

Moieties represented by R are substituted or non-substituted moieties selected from the group consisting of hydroxamic acid, tetrazole, nitro, thiocarboxylic acid, amide, sulfonic acid, sulfonamide, phosphonate, boronic acid, 4-linked 3-hydroxycyclobut-3-ene-1,2-dione, an oxo-oxadiazole-like moiety with optional sulfur-substitutions, and moieties having a general formula II or III. 4-linked 3-hydroxycyclobut-3-ene-1,2-dione resembles squaric acid and is therefore sometimes referred to as a squaric acid moiety.

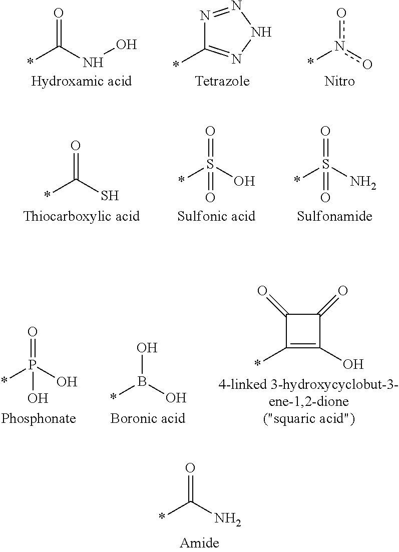

Oxo-oxadiazole-like moieties are based on oxadiazoles. Preferably, the oxo-oxadiazole-like moiety is based on a 3-linked 1,2,4-oxadiazole moiety or on a 5-linked 1,3,4-oxadiazole moiety. An oxo-oxadiazole-like moiety can have none, one, or more of its oxygen atoms substituted by sulfur atoms. Preferred oxo-oxadiazole-like moieties for R are depicted below.

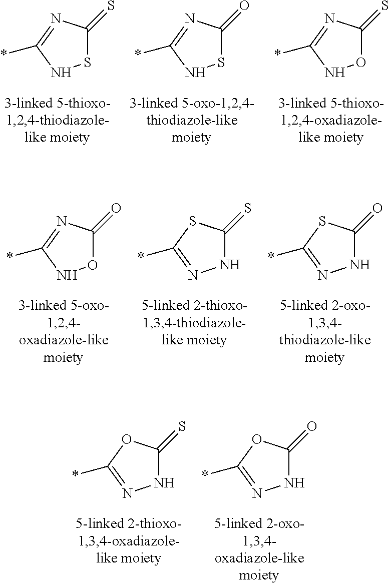

Preferred species of each of these moieties are capable of carrying a net negative charge under physiological conditions. Unsubstituted species of preferred moieties represented by R are depicted below. The most preferred moieties represented by R are hydroxamic acid, amide, thiocarboxylic acid, or tetrazole, each of them unsubstituted. Hydroxamic acid is a most preferred moiety.

Moieties of general formula II or III preferably have at most twenty atoms, fifteen atoms, twelve atoms, ten atoms, or fewer. Moieties of general formula III are preferably of general formula IIIa or of general formula IIIb:

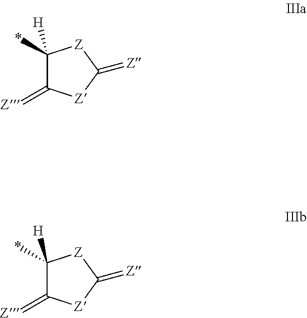

Preferably, R is selected from the group consisting of:

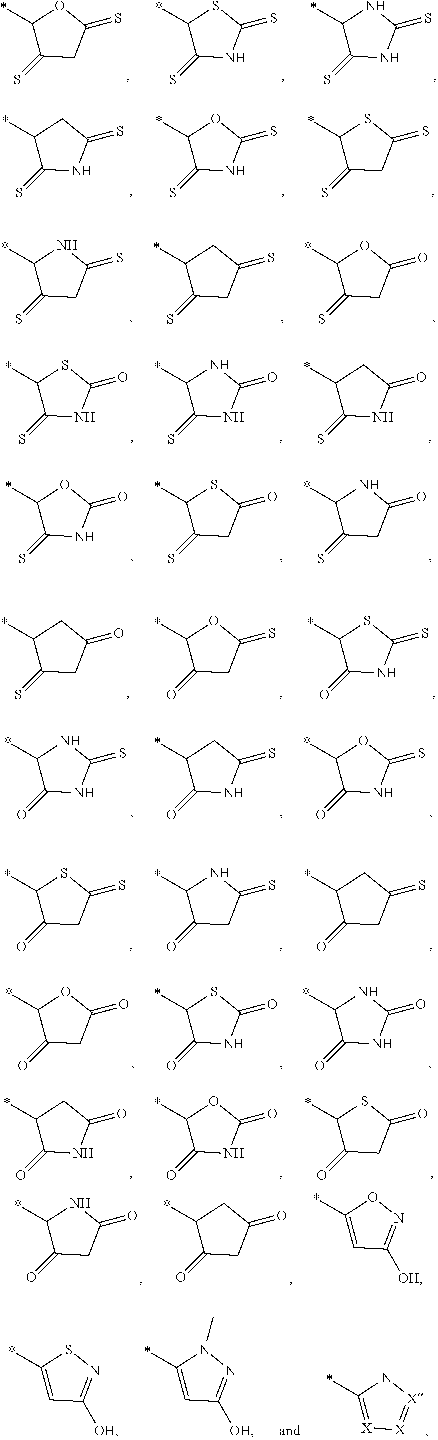

wherein X, X′, and X″ are each individually N, CH, or C—OH. In preferred compounds where R comprises a cyclic moiety, Q is a bond. As described above, moieties that meet the restrictions of general formula III can be of general formula IIIa or of general formula IIIb.

Oxo-oxadiazole-like moieties and moieties of general formula II or III are five-membered heterocycles, and for this reason they can be referred to as five-membered heterocyclic moieties. Tetrazole is a moiety of general formula II wherein X, X′, X″, and Y are N, and tetrazole is therefore also encompassed by this definition. Five-membered heterocyclic moieties are preferred for R. Tetrazole and oxo-oxadiazole-like moieties are preferred five-membered heterocyclic bioisosteres, while tetrazole and 3-linked 5-oxo-1,2,4-oxadiazole-like moiety are most preferred.

In preferred embodiments of this aspect, R is a moiety selected from the group consisting of hydroxamic acid, tetrazole, nitro, thiocarboxylic acid, sulfonic acid, sulfonamide, phosphonate, boronic acid, 4-linked 3-hydroxycyclobut-3-ene-1,2-dione, an oxo-oxadiazole-like moiety with optional sulfur-substitutions, and moieties having a general formula II or III; wherein other variables are as defined above. In more preferred embodiments of this aspect, R is a moiety selected from the group consisting of hydroxamic acid, tetrazole, an oxo-oxadiazole-like moiety with optional sulfur-substitutions, and moieties having a general formula II or III; wherein other variables are as defined above. In even more preferred embodiments of this aspect, R is a moiety selected from the group consisting of hydroxamic acid, tetrazole, and an oxo-oxadiazole-like moiety with optional sulfur-substitutions, wherein other variables are as defined above. In still more preferred embodiments of this aspect, R is a moiety selected from the group consisting of hydroxamic acid, tetrazole, and an oxo-oxadiazole-like moiety, wherein other variables are as defined above. In some most preferred embodiments of this aspect, R is a moiety selected from the group consisting of hydroxamic acid, tetrazole, and 3-linked 5-oxo-1,2,4-oxadiazole-like moiety, wherein other variables are as defined above.

In highly preferred embodiments of this aspect, R is hydroxamic acid. In even more preferred embodiments of this aspect, the compound is of general formula Ia and R is hydroxamic acid. It is even more preferred when such a compound is for use as a medicament, preferably wherein said medicament is for inhibiting HMG-CoA reductase while not inhibiting Complex III.

In preferred embodiments of this aspect, the compound is of general formula Ia, R is hydroxamic acid, and moieties represented by R′ are chosen from the group consisting of R′-simva, R′-lova, R′-meva, R′-prava, R′-atorva, R′-ceriva, R′-fluva, R′-pitava, and R′-rosuva. It is even more preferred when such a compound is for use as a medicament, preferably wherein said medicament is for inhibiting HMG-CoA reductase while not inhibiting Complex III.

In preferred embodiments of this aspect, the compound is of general formula Ia, R is hydroxamic acid, and moieties represented by R′ are chosen from the group consisting of R′-lova, R′-meva, R′-prava, R′-atorva, R′-ceriva, R′-fluva, R′-pitava, and R′-rosuva. It is even more preferred when such a compound is for use as a medicament, preferably wherein said medicament is for inhibiting HMG-CoA reductase while not inhibiting Complex III.

In preferred embodiments of this aspect, the compound is of general formula Ia, R is hydroxamic acid, and moieties represented by R′ are chosen from the group consisting of R′-atorva, R′-ceriva, R′-fluva, R′-pitava, and R′-rosuva. It is even more preferred when such a compound is for use as a medicament, preferably wherein said medicament is for inhibiting HMG-CoA reductase while not inhibiting Complex III.

In preferred embodiments of this aspect, the compound is of general formula Ia, R is hydroxamic acid, and moieties represented by R′ are chosen from the group consisting of R′-simva, R′-lova, R′-meva, and R′-prava. It is even more preferred when such a compound is for use as a medicament, preferably wherein said medicament is for inhibiting HMG-CoA reductase while not inhibiting Complex III.

In preferred embodiments of this aspect, the compound is of general formula Ia, R is hydroxamic acid, and moieties represented by R′ are chosen from the group consisting of R′-lova, R′-meva, and R′-prava. It is even more preferred when such a compound is for use as a medicament, preferably wherein said medicament is for inhibiting HMG-CoA reductase while not inhibiting Complex III.

In highly preferred embodiments of this aspect, the compound is of general formula Ia or Ie, preferably Ie, and R is an oxo-oxadiazole-like moiety or of general formula II or III. In even more preferred embodiments of this aspect, the compound is of general formula Ia or Ie, preferably Ie, and R is an oxo-oxadiazole-like moiety or tetrazole. In more preferred embodiments of this aspect, the compound is of general formula Ia or Ie, preferably Ie, and R is a 3-linked 5-oxo-1,2,4-oxadiazole-like moiety or tetrazole. It is even more preferred when such a compound is for use as a medicament, preferably wherein said medicament is for inhibiting HMG-CoA reductase while not inhibiting Complex III.

In some preferred embodiments of this aspect, R′ is R′-simva, Q is —CH₂—, and R is nitrile, hydroxamic acid, tetrazole, or an oxo-oxadiazole-like moiety. In other preferred embodiments of this aspect, R′ is R′-simva, Q is a bond, and R is tetrazole or an oxo-oxadiazole-like moiety. In further preferred embodiments of this aspect, R′ is R′-simva, and either Q is a bond while R is tetrazole or an oxo-oxadiazole-like moiety, or Q is —CH₂— while R is nitrile, hydroxamic acid, tetrazole, or an oxo-oxadiazole-like moiety. It is even more preferred when such compounds are for use as a medicament, preferably wherein said medicament is for inhibiting HMG-CoA reductase while not inhibiting Complex III.

In some preferred embodiments of this aspect, R′ is R′-simva, Q is —CH₂—, and R is hydroxamic acid, tetrazole, or an oxo-oxadiazole-like moiety. In further preferred embodiments of this aspect, R′ is R′-simva, and either Q is a bond while R is tetrazole or an oxo-oxadiazole-like moiety, or Q is —CH₂— while R is hydroxamic acid, tetrazole, or an oxo-oxadiazole-like moiety. It is even more preferred when such compounds are for use as a medicament, preferably wherein said medicament is for inhibiting HMG-CoA reductase while not inhibiting Complex III.

In some preferred embodiments of this aspect, R′ is R′-rosuva, Q is —CH₂—, and R is amide, hydroxamic acid, tetrazole, or an oxo-oxadiazole-like moiety. In other preferred embodiments of this aspect, R′ is R′-rosuva, Q is a bond, and R is tetrazole or an oxo-oxadiazole-like moiety. In further preferred embodiments of this aspect, R′ is R′-rosuva, and either Q is a bond while R is tetrazole or an oxo-oxadiazole-like moiety, or Q is —CH₂— while R is amide, hydroxamic acid, tetrazole, or an oxo-oxadiazole-like moiety. In some embodiments of this aspect, R′ is R′-rosuva, and either Q is a bond while R is tetrazole or an oxo-oxadiazole-like moiety, or Q is —CH₂— while R is amide or hydroxamic acid. It is even more preferred when such compounds are for use as a medicament, preferably wherein said medicament is for inhibiting HMG-CoA reductase while not inhibiting Complex III.

The skilled person understands that while all moieties that can be represented by R as described above are depicted herein in their neutral form, they can occur as charged moieties nonetheless. For example, while —COOH is commonly understood to be an acceptable representation of a carboxylic acid, this moiety can be present as a —COOH moiety or as a —COO⁻ moiety, depending on the conditions. Consequently, compounds according to the invention, may be the compounds as depicted or may be a pharmaceutically acceptable salts of such a compound according to the invention. As a non-limiting example, when R is sulfonic acid, the corresponding sodium sulfonate is also encompassed by the invention.

Moieties represented by R′ can be substituted or unsubstituted. Preferred moieties represented by R′ are chosen from the group consisting of R′-simva, R′-lova, R′-meva, R′-prava, R′-rosuva, and R′-pitava as depicted here below. More preferred moieties represented by R′ are R′-simva, R′-rosuva, and R′-pitava without further substituents.

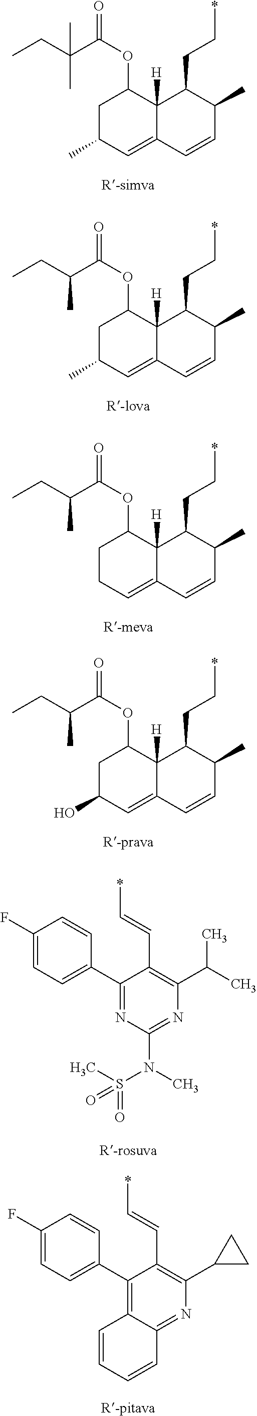

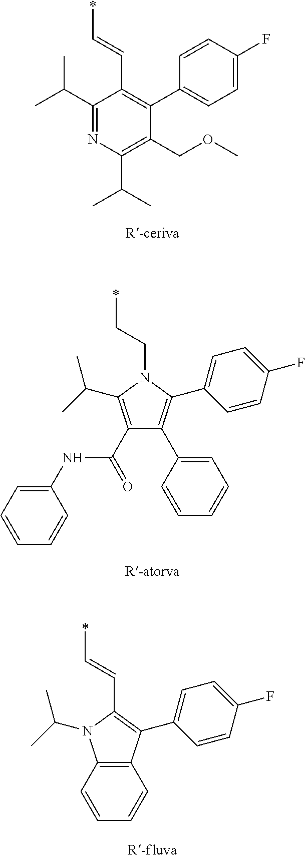 [SMILES: */C=C/c1c(C(C)C)nc(C(C)C)c(COC)c1-c1ccc(F)cc1 ; *CCn1c(-c2ccc(F)cc2)c(-c2ccccc2)c(C(=O)Nc2ccccc2)c1C(C)C ; */C=C/c1c(-c2ccc(F)cc2)c2ccccc2n1C(C)C]

As is clear to a skilled person, all the moieties that are represented by R′ together constitute the group of known statins. It follows that the invention therefore also encompasses compounds of general formula I where R′ represents any other moiety that exhibits good binding to the binding pocket of HMG-CoA reductase that is responsible for binding any of the moieties represented by R′. Preferably, such compound is derived from a statin, preferably from a compound selected from the group consisting of simvastatin, rosuvastatin, pitavastatin, atorvastatin, cerivastatin, fluvastatin, lovastatin, mevastatin, and pravastatin, wherein the carboxylic acid moiety has been replaced by a substituted or non-substituted moiety R as defined herein above.

In preferred embodiments of this aspect, moieties represented by R′ are chosen from the group consisting of R′-simva, R′-lova, R′-meva, R′-prava. In preferred embodiments of this aspect, moieties represented by R′ are chosen from the group consisting of R′-atorva, R′-ceriva, R′-fluva, R′-pitava, and R′-rosuva. In more preferred embodiments of this aspect, moieties represented by R′ are chosen from the group consisting of R′-pitava and R′-rosuva.

Preferred compounds of general formula I are presented in table 1. Table 2 presents compounds of general formula I that are more preferred. Compounds of general formula I that are even more preferred are those that are listed in Table 1 or in Table 2, wherein said compounds are of general formula Ia or of general formula Ie.

TABLE 1
preferred compounds of general formula I

| # | R | R′ | Q |
|---|---|---|---|
| 17 | Sulfonic acid | R′-pitava | CH₂ |
| 18 | Sulfonic acid | R′-rosuva | CH₂ |
| 19 | Phosphonate | R′-pitava | CH₂ |
| 20 | Phosphonate | R′-rosuva | CH₂ |
| 21 | Boronic acid | R′-pitava | CH₂ |
| 22 | Boronic acid | R′-rosuva | CH₂ |
| 23 | Sulfonamide | R′-pitava | CH₂ |
| 24 | Sulfonamide | R′-rosuva | CH₂ |
| 25 | Thiocarboxylic acid | R′-pitava | CH₂ |
| 260 | Thiocarboxylic acid | R′-rosuva | CH₂ |
| 27 | Amide | R′-pitava | CH₂ |
| 28 | Amide | R′-rosuva | CH₂ |
| 29 | 3-linked 5-thioxo-1,2,4-oxadiazole-like moiety | R′-pitava | bond |
| 30 | 3-linked 5-thioxo-1,2,4-oxadiazole-like moiety | R′-rosuva | bond |
| 31 | 3-linked 5-oxo-1,2,4-thiodiazole-like moiety | R′-pitava | bond |
| 32 | 3-linked 5-oxo-1,2,4-thiodiazole-like moiety | R′-rosuva | bond |
| 33 34 | 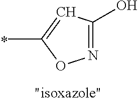 | R′-pitava R′-rosuva | bond bond |
| 35 36 | 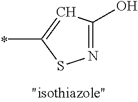 | R′-pitava R′-rosuva | bond bond |
| 37 38 | 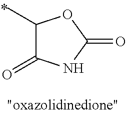 | R′-pitava R′-rosuva | bond bond |
| 39 40 | 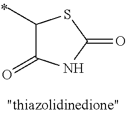 | R′-pitava R′-rosuva | bond bond |
| 41 42 | 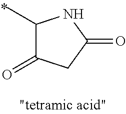 | R′-pitava R′-rosuva | bond bond |
| 43 44 | 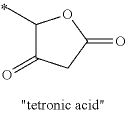 | R′-pitava R′-rosuva | bond bond |
| 45 46 | 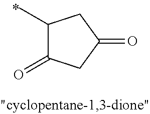 | R′-pitava R′-rosuva | bond bond |
| 47 | Squaric acid | R′-pitava | bond |
| 48 | Squaric acid | R′-rosuva | bond |

TABLE 2
compounds of general formula I that are more preferred

| # | R | R′ | Q |
|---|---|---|---|
| 1 | tetrazole | R′-pitava | CH₂ |
| 2 | tetrazole | R′-pitava | bond |
| 3 | tetrazole | R′-rosuva | CH₂ |
| 4 | tetrazole | R′-rosuva | bond |
| 5 | nitro | R′-pitava | CH₂ |
| 6 | nitro | R′-pitava | bond |
| 7 | nitro | R′-rosuva | CH₂ |
| 8 | nitro | R′-rosuva | bond |
| 9 | hydroxamic acid | R′-pitava | CH₂ |
| 10 | hydroxamic acid | R′-pitava | bond |
| 11 | hydroxamic acid | R′-rosuva | CH₂ |
| 12 | hydroxamic acid | R′-rosuva | bond |
| 13 | 3-linked 5-oxo-1,2,4-oxadiazole-like moiety | R′-pitava | CH₂ |
| 14 | 3-linked 5-oxo-1,2,4-oxadiazole-like moiety | R′-pitava | bond |
| 15 | 3-linked 5-oxo-1,2,4-oxadiazole-like moiety | R′-rosuva | CH₂ |
| 16 | 3-linked 5-oxo-1,2,4-oxadiazole-like moiety | R′-rosuva | bond |

Preferably, Q is a bond for those compounds of general formula I where R is a cyclic moiety such as tetrazole, squaric acid, an oxo-oxadiazole-like moiety with optional sulfur-substitutions, or a moiety of general formula II or of general formula III.

The compounds according to the invention inhibit HMG-CoA reductase. This enzyme has been presented herein above. Inhibition of HMG-CoA reductase activity ultimately leads to reduced biosynthesis of cholesterol. More directly, such inhibition leads to reduced biosynthesis of mevalonic acid, which is (3R)-3,5-Dihydroxy-3-methylpentanoic acid. Inhibition of HMG-CoA reductase can be assessed using assays known in the art, preferably as described in the examples herein.

Inhibition of an enzyme relates to a reduction of the activity of said enzyme. Inhibition is preferably expressed as the remaining percentage of activity of an enzyme that is detected while an amount, preferably a known amount, of inhibitor is present, as compared to the activity of said enzyme without said inhibitor present, which is often the same as the activity of a control sample where only vehicle was added. The term “vehicle” relates to the sum of excipients such as solvents that was used. Inversely, inhibition can also be expressed as the amount of activity that is suppressed in this way. This amounts to 100% minus the percentage of remaining activity as depicted here above. Another preferred way of expressing inhibition is through the half-maximal inhibitory concentration (IC₅₀) value. This represents the effectiveness of a substance in inhibiting a specific biological or biochemical function, in this case HMG-CoA reductase activity. In the present context, IC₅₀ indicates how much of a compound according to the invention is needed to inhibit HMG-CoA reductase by half. IC₅₀ is commonly used as a measure of antagonist drug potency in pharmacological research and represents the concentration of a drug that is required for 50% inhibition in vitro.

In the context of this invention, a compound according to the invention is said to inhibit an enzyme if an effective dose of the compound reduces the activity of said enzyme to a remaining 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, or 0% of the activity of the enzyme when assayed under identical conditions without a compound according to the invention. This can mean that no detectable activity is present in the case of inhibition.

Compounds according to the invention do not inhibit Complex III. This is to be understood in the proper context: when present at an excessive concentration, each compound could be said to inhibit Complex III at least somewhat, and when present at a negligible concentration, each compound could be said to not inhibit Complex III. Accordingly, a compound according to the invention only demonstrates low inhibition of Complex III. Low inhibition is to be understood as a level of inhibition that is lower than the level of inhibition that would be caused by an analogue of a compound of the invention. More particularly, for each specific R′ moiety and with said moiety kept constant, a compound of general formula I is said to not inhibit Complex III when it shows less inhibition of Complex III than an equal amount of a compound of general formula IV or of general formula V, particularly of general formula V. Therefore, a compound of general formula I is said to not inhibit Complex III. As a non-limiting example, a compound of general formula I where R′ is R′-simva is said to not inhibit Complex III, because it shows significantly less inhibition of Complex III than the free acid or corresponding lactone of simvastatin, which are compounds of general formula IV where R′ is R′-simva (free acid) or of general formula V where R′ is R′-simva (lactone). Accordingly, there is provided a preferred compound that inhibits 3-hydroxy-3-methylglutaryl-coenzyme A (HMG-CoA) reductase while showing reduced inhibition of mitochondrial complex III, wherein said compound has the general formula I as defined above. More preferably, said compound is a compound of the general formula I as defined above which feature an R′ moiety as defined above, wherein said compound inhibit 3-hydroxy-3-methylglutaryl-coenzyme A (HMG-CoA) reductase while showing less inhibition of mitochondrial complex III than compounds of general formula IV or of general formula V with the same R′ moiety. In this document, whenever a compound is said to not inhibit Complex III, it is to be understood that said compound does not induce myopathy, or that said compound induces less myopathy.

Herein, a compound according to the invention can be said to not inhibit Complex III if the remaining Complex III activity is 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the activity of the enzyme when assayed under identical conditions without a compound according to the invention. Also, a compound according to the invention of general formula I with a specific R′ moiety can be said to not inhibit Complex III when the inhibition by an analogue with identical R′ moiety and that has general formula IV or general formula V is 1%, 5%, 10%, 15%, 20%, 25%, 30%, 35%, 40%, 50%, 55%, 60%, 65%, 70%, 75%, 80%, 85%, 90%, 95%, or 100%, or more greater than the inhibition of Complex III shown by said compound according to the invention of general formula I.

Composition

In a second aspect of the invention, a composition comprising a compound according to the first aspect is provided. A compound according to the first aspect of the invention can also be a pharmaceutically acceptable salt of said compound. In a preferred embodiment, this composition according to the invention is a pharmaceutical composition. A composition according to the invention preferably further comprises an excipient, more preferable a pharmaceutically acceptable excipient. Preferred excipients are adjuvants, binders, desiccants, or diluents.

A composition according to the invention can be a parenteral composition. For parenteral compositions, preferred excipients are pH regulators, buffering agents, osmolality regulators such as salts or sugars, and surfactants or other agents that help prevent aggregation.

Further preferred compositions additionally comprise ezetimibe, bile-acid resins, fibric acid derivatives, apolipoprotein B (Apo B) inhibitors, angiopoietin-like 3 (ANGPTL3) inhibitors, niacin or its derivatives, amlodipine preferably as amlodipine besylate, aspirin, ascal, clopidogrel, warfarin, beta-blockers, CEPT inhibitors, bempedoic acid, PPAR-delta agonists, acetyl coenzyme A carboxylase inhibitors, or angiotensin-converting-enzyme inhibitors (ACE-inhibitors) such as perindopril, captopril, enalapril, lisinopril, or ramipril.

Preferably, a composition according to the invention is provided in the form of a tablet, soft or hard capsule, ampoule, solution for injection, emulsion for injection, suspension for injection, solution for inhalation, emulsion for inhalation, suspension for inhalation, cream, or ointment. Such composition according to the invention is preferably a pharmaceutical composition.

Medical Use

In a third aspect of the invention is provided a compound as described above, or a pharmaceutical composition as described above, for use as a medicament. Accordingly, an embodiment of this aspect provides a compound according to the invention for use as a medicament. This aspect also provides a composition according to the invention, preferably a pharmaceutical composition according to the invention for use as a medicament. A compound or composition for use as a medicament is preferably for use in the treatment, prevention, or delay of a condition or disease.

Preferably, the use as a medicament is for the treatment, prevention, or delay of a cardiovascular disease, hypercholesterolemia, hypertriglyceridemia, a metabolic disorder, inflammation, nephropathy, chronic obstructive pulmonary disease (COPD), dementia, venous thromboembolism, vascular inflammation, contrast-induced nephropathy, acute kidney injury, pancreatitis, sepsis, acute respiratory distress syndrome, dengue, multiple organ dysfunction syndrome, vitiligo, cancer (particularly ovarian cancer, hematologic cancer, liver cancer, prostate cancer, colorectal cancer, or pancreatic cancer), and/or Alzheimer's disease or neurodegenerative disease in a subject, wherein said use preferably comprises administering to the subject an effective amount of either a compound according to the invention, or the pharmaceutical composition according to the invention. Administration of such compound or composition according to the invention can be achieved by any method known in the art, as defined later herein. Herein, an effective dose of a compound according to the invention or composition according to the invention is a dose that can assert a desired effect, such as improving a symptom of a disorder, or changing a parameter associated with a disorder, or more specifically such as inhibiting HMG-CoA reductase while not inhibiting Complex III.

In a fourth aspect of the invention, there is provided the use of a compound according to the invention, of a composition according to the invention or of a pharmaceutical composition according to the invention, in the manufacture of a medicament. Preferably, said use is for the manufacture of a medicament for the treatment, prevention, or delay of a cardiovascular disease, hypercholesterolemia, hypertriglyceridemia, a metabolic disorder, inflammation, nephropathy, COPD, dementia, venous thromboembolism, vascular inflammation, contrast-induced nephropathy, acute kidney injury, pancreatitis, sepsis, acute respiratory distress syndrome, dengue, multiple organ dysfunction syndrome, vitiligo, cancer (particularly ovarian cancer, hematologic cancer, liver cancer, prostate cancer, colorectal cancer, or pancreatic cancer), and/or Alzheimer's disease or neurodegenerative disease in a subject. Preferably, said use comprises administering to the subject an effective amount of either a compound or a pharmaceutical composition according to the invention. Administration of such compound or composition according to the invention can be achieved by any method known in the art, as defined later herein.

In a fifth aspect, there is provided a method for the treatment, prevention, or delay of a cardiovascular disease, hypercholesterolemia, hypertriglyceridemia, a metabolic disorder, inflammation, nephropathy, COPD, dementia, venous thromboembolism, vascular inflammation, contrast-induced nephropathy, acute kidney injury, pancreatitis, sepsis, acute respiratory distress syndrome, dengue, multiple organ dysfunction syndrome, vitiligo, cancer (particularly ovarian cancer, hematologic cancer, liver cancer, prostate cancer, colorectal cancer, or pancreatic cancer), and/or Alzheimer's disease or neurodegenerative disease in a subject, said method comprising administering to the subject an effective amount of either a compound according to the invention, a composition according to the invention or a pharmaceutical composition according to the invention.

Compositions and pharmaceutical compositions according to the invention may be manufactured by processes well known in the art; e.g. by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/microparticulate powders, or any other shape or form. Compositions for use in accordance with the invention thus may be formulated in conventional manner using one or more physiologically acceptable carriers comprising excipients and auxiliaries that facilitate processing of the active compounds into preparations that can be used pharmaceutically. Proper formulation is dependent on the route of administration chosen.

For injection, a compound according to the invention may be formulated in aqueous formulations, which can for example be solutions, emulsions, suspensions, or liposomal formulations. Aqueous formulations are preferably in pharmaceutically acceptable and/or physiologically compatible buffers such as Hanks's solution, Ringer's solution, or physiological saline buffer. For transmucosal administration, penetrants appropriate to the barrier to be permeated are used in the formulation. Such penetrants are generally known in the art.

Formulations that promote penetration of the epidermis are known in pharmacology, and can find use in the treatment of many skin conditions, such as, but not limited to, psoriasis and fungal infections. Formulations that promote penetration of the epidermis and underlying layers of skin are also known, and can be used to apply compositions of the invention to, for example, underlying muscle or joints. In some preferred therapeutic embodiments, formulation comprising compositions according to the invention that deliver compounds for alleviation of rheumatoid- or osteo-arthritis can be administered by applying a cream, ointment, or gel to the skin overlying the affected joint. Creams are known in the art, and are generally a viscous aqueous emulsion of oil and/or fat, wherein said emulsion comprises at least one pharmaceutical agent. Ointments are known in the art, and are generally viscous preparations of oils and/or fats, which comprise at least one pharmaceutical agent.

Oral and parenteral administration may be used. If so, the compound or composition according to the invention can be formulated readily by combining a compound or composition according to the invention with pharmaceutically acceptable carriers well known in the art, or by using a compound or composition according to the invention as a food additive. Such strategies enable the compounds or compositions according to the invention to be formulated as tablets, pills, dragees, capsules, liquids, gels, syrups, slurries, suspensions and the like, for oral ingestion by a subject to be treated. Preparations or pharmacological preparations for oral use can be made with the use of a solid excipient, optionally grinding the resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinyl pyrrolidone, agar, or alginic acid or a salt thereof such as sodium alginate. Additionally, coformulations may be made with uptake enhancers known in the art.

Dragée cores are provided with suitable coatings. For this purpose, concentrated sugar solutions may be used, which may optionally contain gum arabic, talc, PVP, carbopol gel, polyethylene glycol, and/or titanium dioxide, lacquer solution, and suitable organic solvents or solvent mixtures. Polymethacrylates can be used to provide pH-responsive release profiles so as to pass the stomach. Dyestuffs or pigments may be added to the tablets or dragée coatings for identification or to characterize different combinations of active compound doses.

Pharmaceutical compositions, which can be administered orally include push-fit capsules made of gelatin, as well as soft, sealed capsules made of gelatin and a plasticizer, such as glycerol or sorbitol. The push-fit capsules can contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compounds or compositions according to the invention may be administered in the form of tablets or lozenges formulated in a conventional manner.

For administration by inhalation, the compounds and compositions according to the invention can be conveniently delivered in the form of an aerosol spray presentation from pressurized packs or a nebulizer, with the use of a suitable propellant, e.g., dichlorodifluoromethane, trichlorofluoromethane, dichloro-tetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and optionally a suitable powder base such as lactose or starch.

The compound or composition according to the invention may be formulated for parenteral administration by injection, e.g., by bolus injection or continuous infusion. In this way it is also possible to target a particular organ, tissue, tumor site, site of inflammation, etc. Formulations for infection may be presented in unit dosage form, e.g., in ampoules or in multi-dose container, with an added preservative. The compositions may take such forms as suspensions, solutions or emulsions in oily or aqueous vehicles, and may contain formulatory agents such as suspending, stabilizing and/or dispersing agents. Additional preferred excipients are pH regulators, buffering agents, osmolality regulators such as salts or sugars, and surfactants or other agents that help prevent aggregation.

Compositions or pharmaceutical compositions for parenteral administration include aqueous solutions of the compositions in water soluble form. Additionally, suspensions of the compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

Alternatively, one or more components of the composition may be in powder form for constitution with a suitable vehicle, e.g., sterile pyrogen-free water, or water for injection (WFI), before use.

The compositions may also be formulated in rectal compositions such as suppositories or retention enemas, e.g., containing conventional suppository bases such as cocoa butter or other glycerides.

In addition to the formulations described previously, the composition according to the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil), or as part of a solid or semi-solid implant that may or may not be auto-degrading in the body, or ion exchange resins, or one or more components of the composition can be formulated as sparingly soluble derivatives, for example, as a sparingly soluble salt. Examples of suitable polymeric materials are known to the person skilled in the art and include PLGA, PLA, PGA, and polylactones such as polycaproic acid.

The compositions or pharmaceutical compositions according to the invention also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Compounds, compositions and/or pharmaceutical compositions for use in the invention include compounds and compositions wherein the active ingredients are contained in an amount effective to achieve their intended purposes. More specifically, a therapeutically effective amount means an amount of compound effective to prevent, alleviate or ameliorate symptoms of disease or prolong the survival of the subject being treated, more particularly a dose effective to inhibit HMG-CoA reductase. Determination of a therapeutically effective amount is within the capability of those skilled in the art, especially in light of the detailed disclosure provided herein.

Toxicity and therapeutic efficacy of a compound or composition according to the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio between LD₅₀ and ED₅₀. Compounds or compositions exhibiting high therapeutic indices are preferred. The data obtained from these cell culture assays and animal studies can be used in formulating a range of dosage for human use. The dosage may vary within this range depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics” Ch. 1 p. 1).

The amount of compound or composition administered will, of course, be dependent on the subject being treated, on the subject's weight, the severity of the affliction, the manner of administration and the judgment of the prescribing physician.

A pharmaceutical composition that comprises a compound or a composition according to the invention in combination with a further therapeutic compound can be supplied such that the compound and one or more of the composition components, and the further therapeutic compound are in the same container, either in solution, in suspension, or in powder form. The compound or composition according to the invention can also be provided separately from one or more of the further molecules, and can be mixed with one or more of the further molecules prior to administration. Various packaging options are possible and known to the ones skilled in the art, depending, among others, on the route and mechanism of administration. For example, where the compound according to the invention is supplied separately from one or more of the further therapeutic compounds, the compositions may, if desired, be presented in a pack having more than one chamber, and in which a barrier can be ruptured, ripped, or melted to provide mixing of the compound or composition according to the invention with the further therapeutic compound. Alternatively, two separately provided elements can be mixed in a separate container, optionally with the addition to one or more other carriers, solutions, etc. One or more unit dosage forms containing the further therapeutic compound can be provided in a pack. The pack or dispenser device may be accompanied by instructions for administration. Compositions comprising a compound according to the invention formulated in a compatible pharmaceutical carrier may also be prepared, placed in an appropriate container, and labeled for treatment of an indicated condition. Suitable conditions indicated on the label may include any disease which may be treated or prevented or diagnosed using the compositions according to the invention.

Good therapeutic results can be achieved through adoption of a dosage regime or of incidental administration of low doses of compounds or compositions according to the invention. The invention allows for the further prevention of side effects through its use of low doses. HMG-CoA reductase inhibitors known in the prior art are often only effective at doses that incur significant side effects. In relation to this, the invention allows for dosage regimes that involve an intake schedule featuring intake moments that occur multiple times a day, three times a day, two times a day, daily, weekly, twice a week, preferably six, five, four, or three times a week, while freeing a patient from experiencing side effects, independent of which dosage regime was selected. This promotes therapy adherence and drug fidelity.

Screening Method

In a sixth aspect of the invention, a screening method is provided. This method according to the invention is a method for identifying statin analogues that exhibit a reduced level of mitochondrial complex III inhibition, and comprises the following steps:
i) contacting said analogue, or a composition comprising said analogue, with mitochondrial complex III,
ii) analyzing the level of inhibition of mitochondrial complex III activity by said analogue or by said composition.

Definitions for inhibition, reduced inhibition, and mitochondrial complex III have been provided earlier herein. As is known in the art, an analogue is a compound that closely resembles a different compound that it is analogous to. As a non-limiting example, a compound of the invention with general formula I where R′ is R′-simva and where R is hydroxamic acid (as depicted in FIG. 1B) can be said to be an analogue of simvastatin (as depicted in FIG. 1A), which is a compound of general formula IV or V where R′ is R′-simva. Effectively, the difference between these two compounds is limited to the choice of R. In general, compounds of general formula I can be said to be analogues of compounds of general formula IV or of general formula V. This is especially apt when R′ is amongst the general formulae.

In this aspect, whenever reference is made to an analogue, it is to be understood that a composition comprising said analogue is also encompassed.

In the context of the invention, contacting a compound or a composition with Complex III could be seen as part of an assay. A skilled person will know how to perform such an assay. This can comprise adding such a compound or composition to a medium in which Complex III is comprised. This can involve a cell that expresses Complex III. This can also comprise adding such a compound or composition to a medium, buffer, or solution in which such a cell is suspended, or which covers such a cell. Other preferred methods of contacting Complex III with a compound or composition comprise exposing Complex III to a material comprising a compound or composition according to the invention. A preferred method is to contact a compound or composition with Complex III in a mitochondrial fraction system, where Complex III is present in its native membrane. The examples provide additional guidance in this respect.

Similarly, the analysis of the level of inhibition of Complex III activity by the analogue or by the composition comprising said analogue could be seen as part of an assay. A skilled person will know how to assess the inhibition by the analogue or by the composition comprising said analogue. The examples provide additional guidance in this respect.

Preferably, the screening method according to the invention comprises an additional step. A preferred additional step is as follows:
iii) optionally or non-optionally comparing said level of inhibition to a reference value.

As will be evident to a skilled reader, a reference value can be chosen from amongst a few options. A preferred reference value is a fixed value that represents a standard level of inhibition. This can be the statistical average inhibition that is known to be exhibited by a related class of compounds or compositions. For example, a literature value. A good choice for a fixed value would be a value that is known for a compound that the analogue is analogous to. A skilled person knows that conditions should be identical or closely matched, or knows how to adjust for variation.

A more preferred reference value is a reference value that has been obtained from a control experiment. A control experiment is preferably performed in an identical setup that differs only in the compound that is analyzed, with the understanding that this compound is said analogue in the main experiment, and another compound in the control experiment. Preferably, the control experiment analyzes the inhibition of Complex III by the compound that the analogue is analogous to. For adequate comparison, the level of inhibition is preferably expressed in identical units for each experiment.

Preferably, the screening method according to the invention comprises a further additional step. A preferred further step is:
iv) optionally or non-optionally identifying said analogue as exhibiting a reduced level of mitochondrial complex III inhibition when said comparison reveals a decreased level of inhibition.

When the comparison in optional step iii reveals that the analogue, or the composition comprising said analogue, that is being investigated has a lower inhibition of Complex III than the reference value, it can be concluded that the analogue, or the composition comprising said analogue, has a reduced level of mitochondrial complex III inhibition. This can mean that the analogue has a lower inhibition of Complex III than the compound that is analogous to. When no inhibition is detected, this can also mean that the analogue has a reduced inhibition of Complex III. In this case, the analogue does not inhibit Complex III. Preferred levels of inhibition are defined herein above.

Preferably, the screening method according to the invention additionally comprises determining whether the analogue inhibits HMG-CoA reductase. It is possible that the analogue is a known inhibitor of HMG-CoA reductase. In this case, it is especially valuable to identify analogues that maintain this ability, or that maintain this ability to levels that are still effective or acceptable. Accordingly, the method preferably comprises the following steps:
a) contacting said analogue with 3-hydroxy-3-methylglutaryl-coenzyme A (HMG-CoA) reductase,
b) analyzing the level of inhibition of HMG-CoA reductase activity by said analogue,
c) optionally or non-optionally comparing said level of HMG-CoA reductase activity to a reference value, and
d) optionally identifying said analogue as inhibiting HMG-CoA reductase activity when step b) reveals a level of inhibition.

Features and definitions are as provided herein above. An analogue or a composition comprising said analogue can be said to inhibit HMG-CoA reductase activity when it reduces the level of HMG-CoA reductase activity as defined earlier herein. In this case, comparison to the compound that the analogue is analogous to is less important, because as it pertains to HMG-CoA reductase activity, comparison to vehicle, or to uninhibited HMG-CoA reductase activity, is very informative for determining the level of HMG-CoA reductase activity. As such, comparison to a reference value is only useful for reference, and is not essential for identifying whether said analogue can actually inhibit HMG-CoA reductase activity.

### General Definitions

In this application, ‘substances’ should be interpreted as molecules, complexes of multiple molecules, oligomers, polymers, polypeptides, proteins, particles, or fragments thereof.

Unsubstituted alkyl groups have the general formula CₙH₂ₙ₊₁ and may be linear or branched. Unsubstituted alkyl groups may also contain a cyclic moiety, and thus have the concomitant general formula CₙH₂ₙ₋₁. Optionally, the alkyl groups are substituted by one or more substituents further specified in this document. Examples of alkyl groups include methyl, ethyl, propyl, 2-propyl, t-butyl, 1-hexyl, 1-dodecyl, etc.

An aryl group comprises six to twelve carbon atoms and may include monocyclic and bicyclic structures. Optionally, the aryl group may be substituted by one or more substituents further specified in this document. Examples of aryl groups are phenyl and naphthyl.

Arylalkyl groups and alkylaryl groups comprise at least seven carbon atoms and may include monocyclic and bicyclic structures. Optionally, the arylalkyl groups and alkylaryl may be substituted by one or more substituents further specified in this document. An arylalkyl group is for example benzyl. An alkylaryl group is for example 4-t-butylphenyl.

Heteroaryl groups comprise at least two carbon atoms (i.e. at least C₂) and one or more heteroatoms N, O, P or S. A heteroaryl group may have a monocyclic or a bicyclic structure. Optionally, the heteroaryl group may be substituted by one or more substituents further specified in this document. Examples of suitable heteroaryl groups include pyridinyl, quinolinyl, pyrimidinyl, pyrazinyl, pyrazolyl, imidazolyl, thiazolyl, pyrrolyl, furanyl, triazolyl, benzofuranyl, indolyl, purinyl, benzoxazolyl, thienyl, phospholyl and oxazolyl. A preferred heteroaryl group is nicotinamide.

Heteroarylalkyl groups and alkylheteroaryl groups comprise at least three carbon atoms (i.e. at least C₃) and may include monocyclic and bicyclic structures. Optionally, the heteroaryl groups may be substituted by one or more substituents further specified in this document.

Where an aryl group is denoted as a (hetero)aryl group, the notation is meant to include an aryl group and a heteroaryl group. Similarly, an alkyl(hetero)aryl group is meant to include an alkylaryl group and a alkylheteroaryl group, and (hetero)arylalkyl is meant to include an arylalkyl group and a heteroarylalkyl group. A C₂-C₂₄ (hetero)aryl group is thus to be interpreted as including a C₂-C₂₄ heteroaryl group and a C₆-C₂₄ aryl group. Similarly, a C₃-C₂₄ alkyl(hetero)aryl group is meant to include a C₇-C₂₄ alkylaryl group and a C₃-C₂₄ alkylheteroaryl group, and a C₃-C₂₄ (hetero)arylalkyl is meant to include a C₇-C₂₄ arylalkyl group and a C₃-C₂₄ heteroarylalkyl group.

Unless stated otherwise, alkyl groups, alkenyl groups, alkenes, alkynes, (hetero)aryl groups, (hetero)arylalkyl groups, alkyl(hetero)aryl groups, alkylene groups, alkenylene groups, cycloalkylene groups, (hetero)arylene groups, alkyl(hetero)arylene groups, (hetero)arylalkylene groups, alkenyl groups, alkynyl groups, cycloalkyl groups, alkoxy groups, alkenyloxy groups, (hetero)aryloxy groups, alkynyloxy groups and cycloalkyloxy groups may be substituted with one or more substituents independently selected from the group consisting of C₁-C₁₂ alkyl groups, C₂-C₁₂ alkenyl groups, C₂-C₁₂ alkynyl groups, C₃-C₁₂ cycloalkyl groups, C₅-C₁₂ cycloalkenyl groups, C₈-C₁₂ cycloalkynyl groups, C₁-C₁₂ alkoxy groups, C₂-C₁₂ alkenyloxy groups, C₂-C₁₂ alkynyloxy groups, C₃-C₁₂ cycloalkyloxy groups, halogens, amino groups, oxo and silyl groups, wherein the silyl groups can be represented by the formula (R²)₃Si—, wherein R² is independently selected from the group consisting of C₁-C₁₂ alkyl groups, C₂-C₁₂ alkenyl groups, C₂-C₁₂ alkynyl groups, C₃-C₁₂ cycloalkyl groups, C₁-C₁₂ alkoxy groups, C₂-C₁₂ alkenyloxy groups, C₂-C₁₂ alkynyloxy groups and C₃-C₁₂ cycloalkyloxy groups, wherein the alkyl groups, alkenyl groups, alkynyl groups, cycloalkyl groups, alkoxy groups, alkenyloxy groups, alkynyloxy groups and cycloalkyloxy groups are optionally substituted, the alkyl groups, the alkoxy groups, the cycloalkyl groups and the cycloalkoxy groups being optionally interrupted by one of more hetero-atoms selected from the group consisting of O, N and S.

When a structural formula or chemical name is understood by the skilled person to have chiral centers, yet no chirality is indicated, for each chiral center individual reference is made to all three of either the racemic mixture, the pure R enantiomer, and the pure S enantiomer. Whenever a fragment of a molecule, often referred to as a moiety, is represented, an asterisk (*) indicates where the represented moiety is linked to the rest of the molecule. This asterisk does not imply an atom, and neither does it convey information about which atom is at the non-moiety side of the bond. All this is known in the art, and is routine practice.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used for treatment or as a medicament, it can also be used for the manufacture of a medicament for treatment.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 0.1% of the value.

All patent and literature references cited in the present specification are hereby incorporated by reference in their entirety.

### FIGURE LEGENDS

FIG. 1. (A) Structure of simvastatin acid (dashed oval: carboxylic acid); (B) Structure of the statin-like structure based on simvastatin acid, where the carboxylic acid moiety was replaced by a hydroxamic acid moiety (the structure has general formula I where R is hydroxamic acid, where R′ is R′-simva, and where Q is —CH₂—); (C) Simvastatin acid x-ray structure (protein light gray, simvastatin carbon atoms dark gray and heteroatoms light gray; H-bonds gray dashes) (D) docking pose of the simvastatin hydroxamic acid bioisostere (protein light gray, hydroxamic acid bioisostere carbon atoms dark gray, heteroatoms light gray).

FIG. 2. (A) Complex III inhibitory activity of a newly synthesized simvastatin derivative shown in FIG. 1B, at 100 μM. Statistical analysis: Complex III enzyme activity values were compared to vehicle control levels using one-way ANOVA with Dunnett's post-hoc correction analysis, ***p<0.001; (B) HMG-CoA reductase (HMGR) activity at 300 nM of simvastatin acid and the newly synthesized simvastatin derivative shown in FIG. 1B; Statistical analysis: HMG-CoA reductase enzyme activity values were compared to vehicle control levels using one-way ANOVA with Dunnett's post-hoc correction analysis, ***p<0.001; (C) HMGR dose-response curve for simvastatin acid (●) and the newly synthesized simvastatin derivative (▪) shown in FIG. 1B. IC₅₀ values for simvastatin and for the analogue shown in FIG. 1B belonging to the curves displayed in panel C are presented in table 3.

FIG. 3. Synthesis of bioisosteres using the lactone form of Rosuvastatin. Compound numbering in bold is consistent with the examples, italic text can refer to the description.

FIG. 4. (A) Nitrile preparation; (B) Synthesis of heterocyclic carboxylic acid mimics. Compound numbering in bold is consistent with the examples, italic text can refer to the description.

FIG. 5. Synthesis of key intermediate 21. Compound numbering in bold is consistent with the examples.

FIG. 6. Synthesis of bioisosteres of general formula (I) where Q is a bond. Compound numbering in bold is consistent with the examples, italic text can refer to the description.

FIG. 7. Synthesis of simvastatin analogues. (A) amide-based bioisosteres; (B) heterocyclic bioisosteres. Compound numbering in bold is consistent with the examples, italic text can refer to the description.

FIG. 8. Synthesis of lovastatin analogues via the lactone form. Compound numbering in bold is consistent with the examples, italic text can refer to the description.

FIG. 9. Synthesis of atorvastatin analogues via the lactone form. Compound numbering in bold is consistent with the examples, italic text can refer to the description.

FIG. 10. Synthesis of fluvastatin analogues via the lactone form. Compound numbering in bold is consistent with the examples, italic text can refer to the description.

### EXAMPLES

The present invention is further described by the following examples which should not be construed as limiting the scope of the invention.

Unless stated otherwise, the practice of the invention will employ standard conventional methods of molecular biology, virology, microbiology or biochemistry. Such techniques are described in Sambrook et al. (1989) Molecular Cloning, A Laboratory Manual (2^{nd} edition), Cold Spring Harbor Laboratory, Cold Spring Harbor Laboratory Press; in Sambrook and Russell (2001) Molecular Cloning: A Laboratory Manual, Third Edition, Cold Spring Harbor Laboratory Press, NY; in Volumes 1 and 2 of Ausubel et al. (1994) Current Protocols in Molecular Biology, Current Protocols, USA; and in Volumes I and II of Brown (1998) Molecular Biology LabFax, Second Edition, Academic Press (UK); Oligonucleotide Synthesis (N. Gait editor); Nucleic Acid Hybridization (Hames and Higgins, eds.).

### Example 1. Synthesis of Simvastatin Hydroxamic Acid Bioisostere

In this example, a compound according to the invention is produced. It has general formula I where Q is —CH₂—, where R is hydroxamic acid, and where R′ is R′-simva. As such, it is also of general formula Ia.

A solution of Simvastatin (20 mg, 0.048 mmol) in tetrahydrofuran (THF) (0.25 mL) was mixed with 50% aqueous hydroxylamine (15 μL, 0.24 mmol) and the reaction mixture was stirred at room temperature overnight. After removal of the solvent under reduced pressure, the crude product was purified by column chromatography using methanol/dichloromethane (10:1, vol:vol). This procedure yielded 17 mg (82%) of simvastatin hydroxamic acid bioisostere as a colorless solid.

¹H-NMR (400 MHz, CDCl₃): δ 5.98 (d, J=9.7 Hz, 1H), 5.77 (dd, J=9.6, 6.2 Hz, 1H), 5.49 (t, J=2.8 Hz, 1H), 5.44 (q, J=3.0 Hz, 1H), 4.23 (tt, J=7.6, 3.8 Hz, 1H), 3.78 (br s, 1H), 2.20-2.49 (m, 6H), 1.98 (ddd, J=14.9, 8.5, 2.6 Hz, 1H), 1.82-1.86 (m, 1H), 1.52-1.61 (m. 6H), 1.18-1.24 (m, 2H), 1.11 (d, J=2.9 Hz, 6H), 1.09 (J=7.4 Hz, 3H), 0.86 (d, J=7.0 Hz, 3H), 0.82 (t, J=7.5 Hz, 3H).

### Example 2. Measuring Complex III Activity Using Mitochondrial Fractions

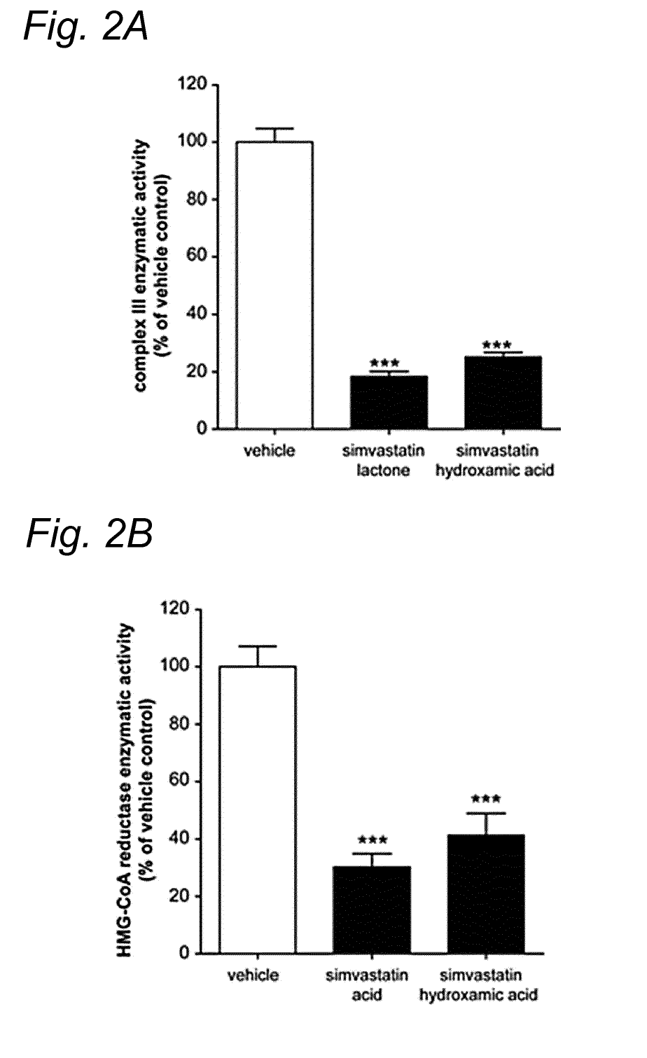

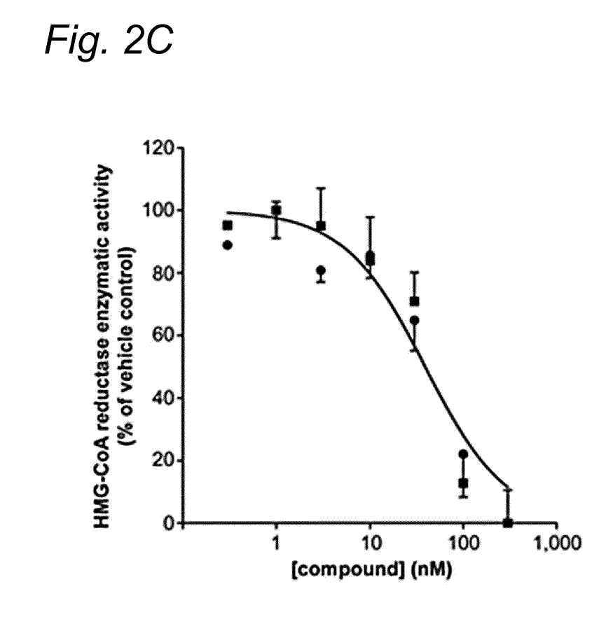

C2C12 pellets of 20·10⁶ cells were snap frozen in liquid nitrogen and kept at −80° C. until use. For preparation of the mitochondrial fractions, tissues and cells were homogenized, resuspended in 10 mM Tris-HCl (pH 7.6), and snap frozen in aliquots as follows: cells were resuspended in 10 mM Tris-HCl and pottered, and sucrose was added (215 mM). The lysate was cleared of unbroken cells by centrifugation (10 minutes 600 g) after which the supernatant containing the mitochondria was pelleted at 14,000 g for 10 minutes, resuspended in 10 mM Tris-HCl (pH 7.6), and snap frozen in aliquots. Complex III enzyme activity was measured spectrophotometrically in duplicate as described before by determination of the cytochrome c reduction at 550 nm (Janssen et al., 2007; Mourmans et al., 1997; Rodenburg, 2011). For these measurements the following substrates were used: decylubiquinol (300 μM) and cytochrome c (50 μM). Background substraction was based on decylubiquinol auto-oxidation rates for FIG. 2, and on antimycin A (2.5 μM) for table 4. The enzyme activity was determined using the linear domain of the curve. These experiments were generally performed in small reaction volumes to increase throughput. Experiments in larger reaction volumes can improve reliability of obtained values, and are planned.

### Example 3. Measuring HMG-CoA Reductase Activity

The human HMG-CoA reductase activity and inhibition assays were performed using the HMG-CoA reductase Assay Kit from Sigma-Aldrich (Sigma CS-1090), containing the catalytic domain of the human HMG-CoA reductase. Enzyme activity was determined spectrophotometrically, measuring the oxidation of NADPH at 340 nm. The reaction mixture contained: 0.13 mM HMG-CoA, HMG-CoA reductase, and 50 mM Tris-HCl, (pH 7.5). After 15 min incubation at 37° C., the reaction was started with the addition of 0.13 mM NADPH, and monitored for 30 min. The enzyme activity was determined using the linear domain of the curve. These experiments were generally performed in small reaction volumes to increase throughput. Experiments in larger reaction volumes can improve reliability of obtained values, and are planned.

### Example 4. Characteristics of Simvastatin Hydroxamic Acid Bioisostere

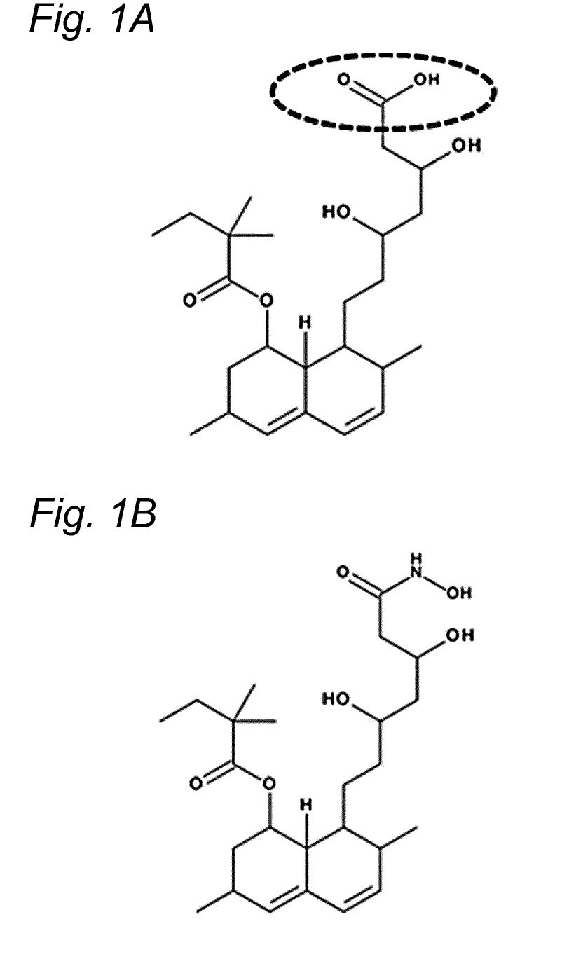

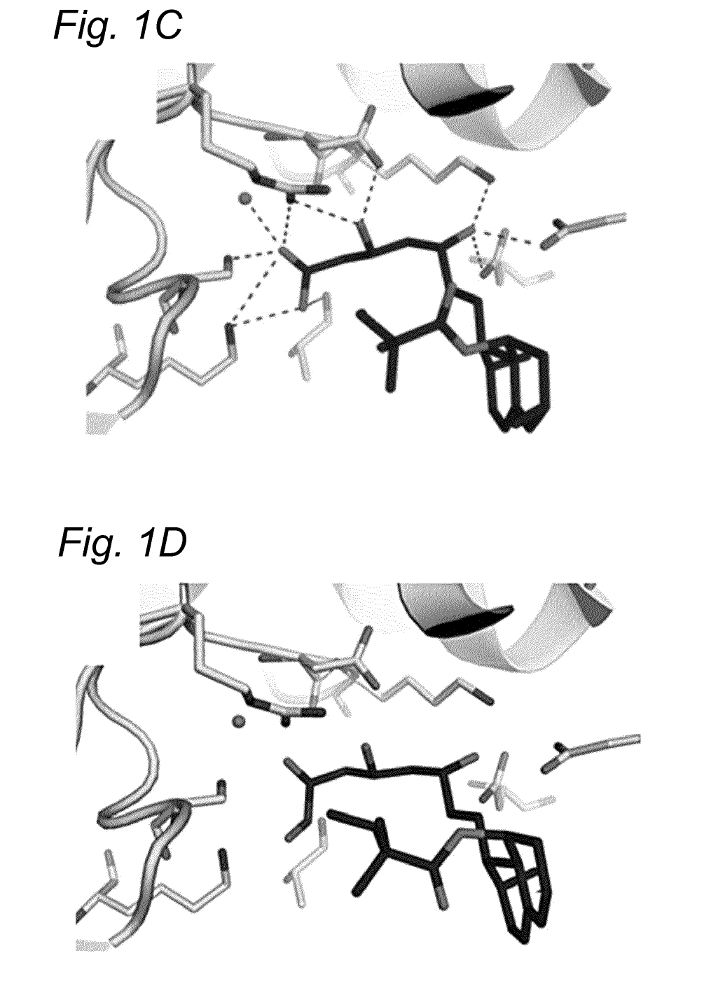

The Complex III inhibitory activity of a newly synthesized simvastatin derivative shown in FIG. 1B was tested at 100 μM, as per the assay described in Example 2. Results of this assay are shown in FIG. 2A. Statistical analysis: Complex III enzyme activity values were compared to vehicle control levels using one-way ANOVA with Dunnett's post-hoc correction analysis, ***p<0.001. The HMG-CoA reductase (HMGR) activity of simvastatin acid and the newly synthesized simvastatin derivative shown in FIG. 1B was also determined, at 300 nM as per the assay described in Example 3. These results are shown in FIG. 2B. Statistical analysis: HMG-CoA reductase enzyme activity values were compared to vehicle control levels using one-way ANOVA with Dunnett's post-hoc correction analysis, ***p<0.001. To further characterize the statin and its analogue, an HMGR dose-response curve was determined for both simvastatin acid (●) and the newly synthesized simvastatin derivative (▪) shown in FIG. 1B. This curve is shown in FIG. 2C. The IC₅₀ values for simvastatin and for the analogue shown in FIG. 1B that belong to the curves displayed in FIG. 2C are shown in table 3:

TABLE 3
mean IC₅₀ values (HMG-CoA reductase)

| | Simvastatin acid | Simvastatin hydroxamic acid |
|---|---|---|
| HMG-CoA reductase | 39 nM | 39 nM |
| mean IC₅₀ | (24 nM-62 nM) | (23 nM-68 nM) |
| (95%-CI) | | |

### Example 5. Rosuvastatin Analogues

The numbering of compounds in this example and those following it is sequential for legibility and not related to any numbering elsewhere in this document.

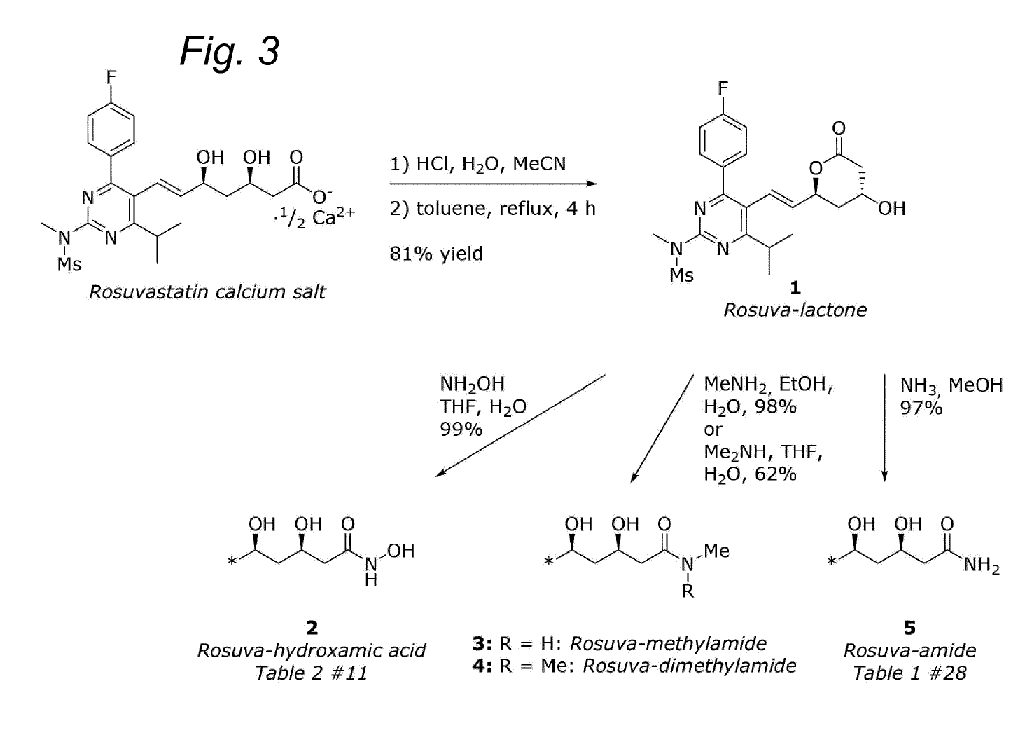

Analogues of rosuvastatin were prepared as depicted in FIG. 3. Lactone 1 was obtained from rosuvastatin calcium salt as described in US2013/296561 A1. During this procedure the material is partially converted into a closely related byproduct, which is removed during the final precipitation of the sequence. This byproduct could originate from epimerization of the C5-hydroxyl group. The ring opening reactions using lactone 1 were performed with 4 different nitrogen nucleophiles, namely hydroxylamine, ammonia, methylamine and dimethylamine. The corresponding rosuvastatin analogues 2, 3, 4, and 5 were obtained in good to excellent yields and purities.

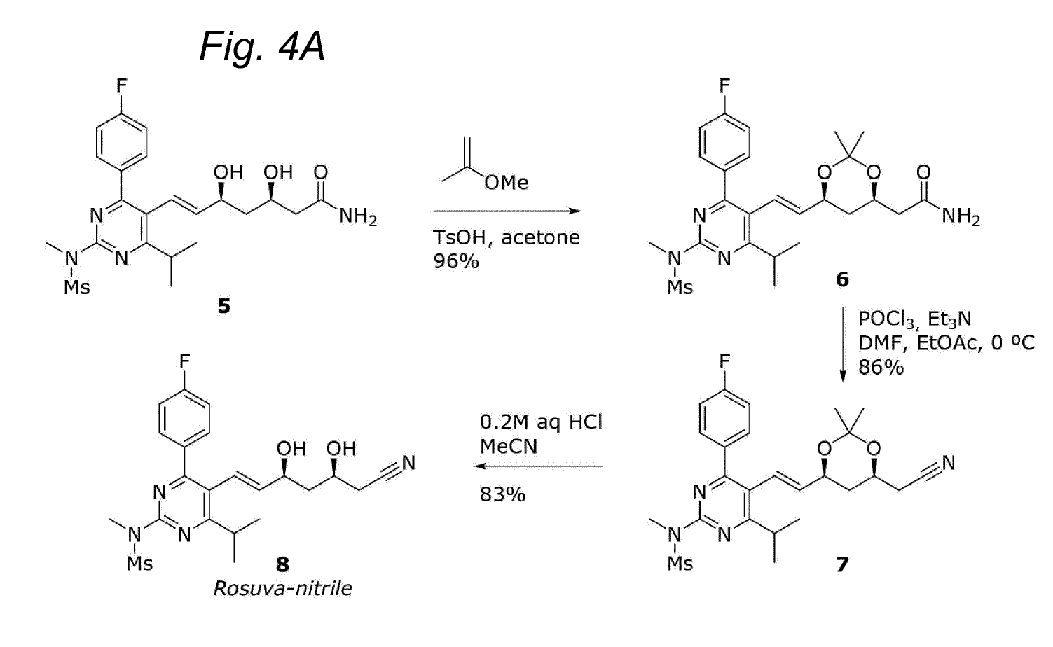

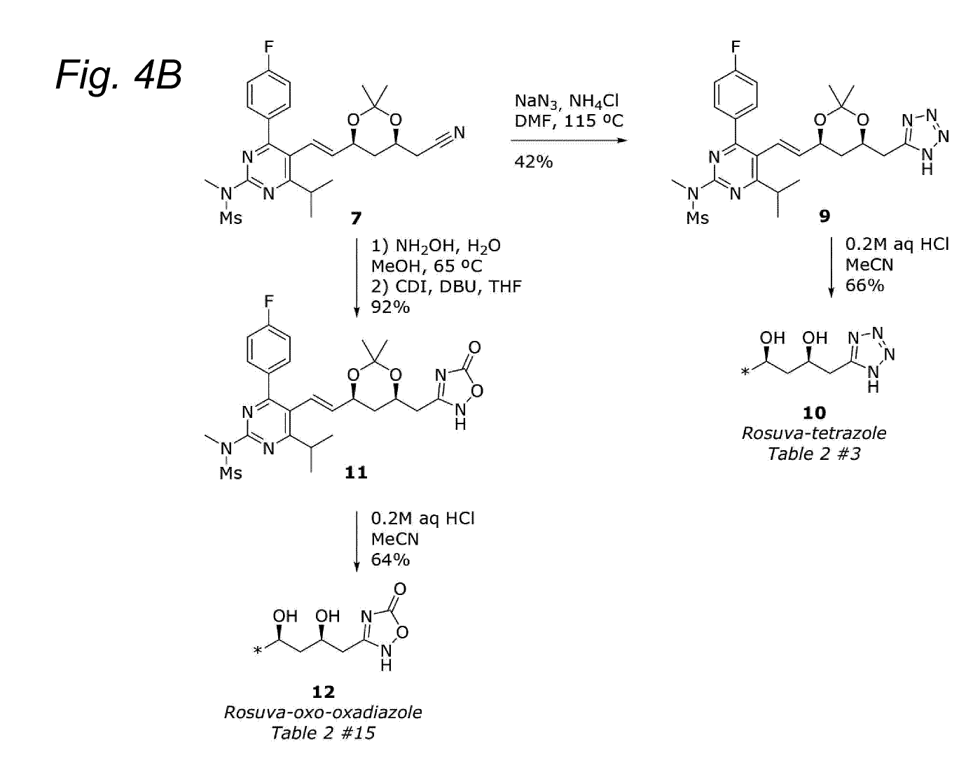

After the diol moiety of 5 was protected as an acetonide (compound 6, FIG. 4a), the amide could be dehydrated by treatment with phosphoryl chloride and N,N-dimethylformamide (DMF). The corresponding nitrile 7 was isolated in a good yield of 86% after purification by column chromatography. After deprotection under acidic conditions rosuvastatin analogue 8 was obtained.

Intermediate 7 was also transformed into tetrazole 9 by treatment with sodium azide and ammonium chloride, which, after acid mediated deprotection was reacted into analogue 10 (FIG. 4b). Reaction of nitrile 7 with hydroxylamine followed by a cyclisation reaction using carbonyldiimidazole (CDI) provided 5-oxo-1,2,4-oxadiazole 9, which also was deprotected to yield substrate 12.

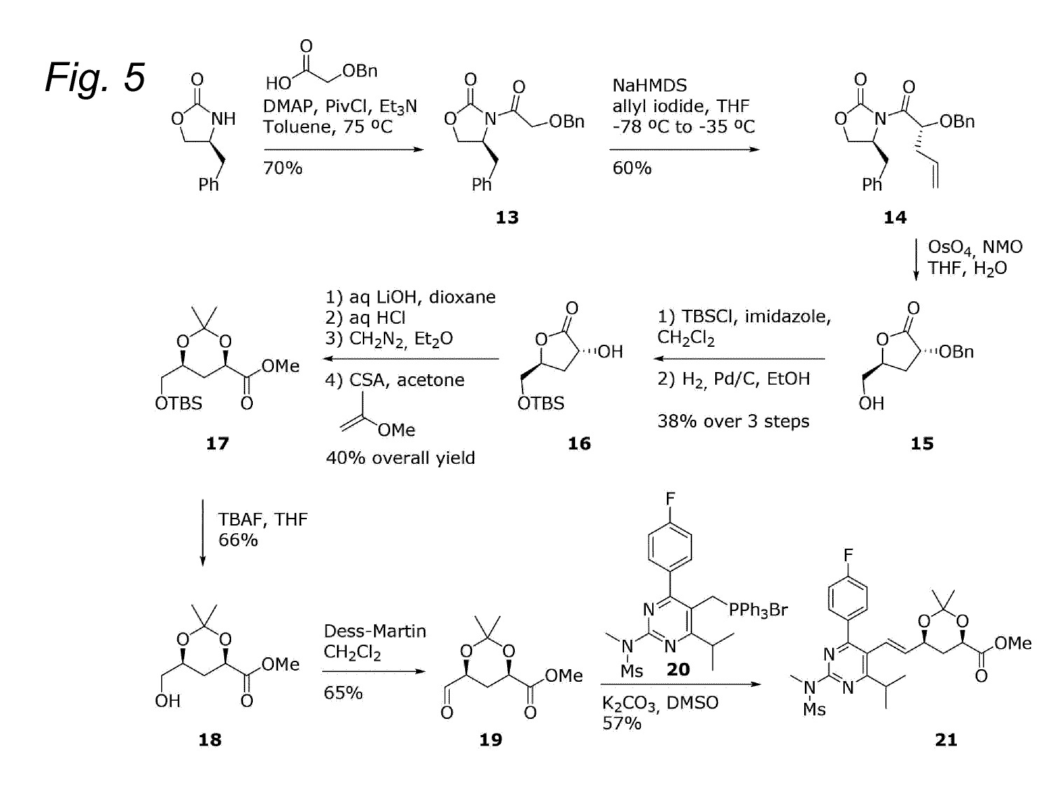

For the preparation of bioisosteres of general formula Ie a synthetic strategy towards key intermediate 21 was developed as described in FIG. 5. The route starts with the synthesis of compound 13, by the coupling of an Evans chiral auxiliary and benzyloxyacetic acid, according to a reported route (Org. Lett. 2010, 12, 3792-3795). The diastereoselective alkylation of 13 provided allyl 14, which upon treatment with osmium tetroxide and NMO yielded alcohol 15. After a protection (TBSCl) and deprotection (Pd/C, H₂) sequence mono-protected lactone 16 was obtained in a moderate yield of 38% over three steps. Hydrolysis of the lactone using aqueous LiOH followed by acidification with aqueous HCl yielded the corresponding carboxylic acid after lyophilization. The acid was converted into the methyl ester by treatment with diazomethane, which was immediately reacted with 2-methoxypropene under acidic conditions to capture the diol moiety to avoid lactone formation. The sequence was described in literature (Tetrahedron Lett. 1987, 28, 1143-1146) and provided methyl ester 17 in a 40% yield. Silyl deprotection by treatment with tetra-n-butylammonium fluoride (TBAF) yielded alcohol 18 and after a Dess-Martin oxidation aldehyde 19 was obtained. A final Wittig reaction between the aldehyde and phosphonium salt 20 (Org. Biomol. Chem., 2016, 14, 1363-1369) provided the desired Rosuvastatin analogue 21 after 11 consecutive steps.

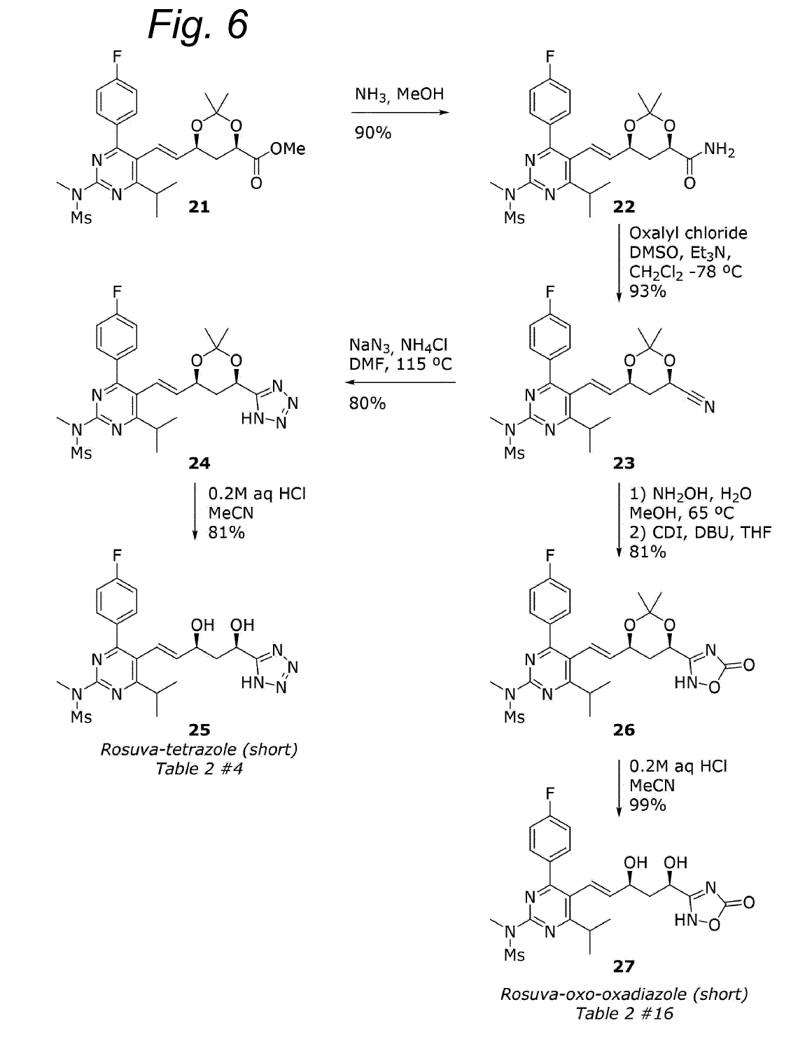

The transformation of methyl ester 21 into two rosuvastatin analogues is described in FIG. 6. Reaction with hydroxylamine provided amide 22, which was dehydrated using oxalyl chloride and dimethyl sulfoxide (DMSO), yielding nitrile 23 in a yield of 93%. This building block was converted into tetrazole 25 and oxo-oxadiazole 27 by applying the same methodology as used for the synthesis of substrates 10 and 12 (FIG. 4b).

### N-(4-(4-fluorophenyl)-5-((E)-2-((2S,4R)-4-hydroxy-6-oxotetrahydro-2H-pyran-2-yl)vinyl)-6-isopropylpyrimidin-2-yl)-N-methylmethanesulfonamide 1

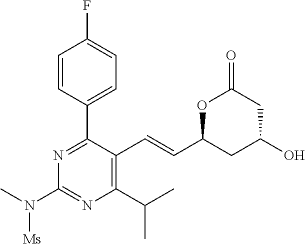 [SMILES: CC(C)c1nc(N(C)S(C)(=O)=O)nc(-c2ccc(F)cc2)c1/C=C/[C@@H]1C[C@@H](O)CC(=O)O1]

This reaction was performed in the dark. Rosuvastatin calcium salt (1 g, 0.99 mmol) was dissolved in acetonitrile (10 mL) and brine (2 mL) was added. The solution was chilled to 0-5° C. and the pH was adjusted to 4.0 using a mixture of 4N HCl (0.5 mL) and brine (1.1 mL). Water (1.5 mL) was added to dissolve the formed salts. EtOAc (12 mL) was added, the aqueous phase was removed and the organic phase was dried with Na₂SO₄, and filtered before concentration under reduced pressure.

The obtained syrup was dissolved in toluene (15 mL) and the mixture was refluxed under Dean-Stark conditions for 4 h. The mixture was cooled to room temperature and stirred for 16 h. The obtained thick suspension was filtered and the gel-like material was washed twice with a small volume of toluene and dried in vacuo to yield 1 as a white solid (750 mg, 81%). ¹H NMR in accordance with literature data (US2013/296561 A1, 2013).

### (3R,5S,E)-7-(4-(4-fluorophenyl)-6-isopropyl-2-(N-methylmethylsulfonamido)pyrimidin-5-yl)-N,3,5-trihydroxyhept-6-enamide 2

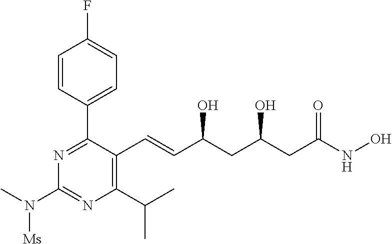 [SMILES: CC(C)c1nc(N(C)S(C)(=O)=O)nc(-c2ccc(F)cc2)c1/C=C/[C@@H](O)C[C@@H](O)CC(=O)NO]

This reaction was performed in the dark. Lactone 1 (43 mg, 0.093 mmol) was dissolved in THF (0.8 mL) and a 50% aqueous solution of hydroxylamine (0.031 mL, 0.464 mmol) was added. The solution was stirred at rt for 2.5 h. Next, the solution was concentrated under reduced pressure in the dark and stripped with CH₂Cl₂ to yield 2 as a white foam. ¹H NMR (400 MHz, CDCl₃): δ 7.63-7.60 (m, 2H), 7.11-7.05 (m, 2H), 6.59 (d, J=15.6 Hz, 1H), 5.44 (dd, J=16.1, 5.6 Hz, 1H), 4.45-4.39 (m, 1H), 4.23-4.14 (m, 1H), 3.56 (s, 3H), 3.51 (s, 3H), 3.35-3.28 (m, 1H), 2.37-2.23 (m, 2H), 1.63-1.45 (m, 2H), 1.25 (d, J=6.7 Hz, 6H).

### (3R,5S,E)-7-(4-(4-fluorophenyl)-6-isopropyl-2-(N-methylmethylsulfonamido)-pyrimidin-5-yl)-3,5-dihydroxy-N-methylhept-6-enamide 3

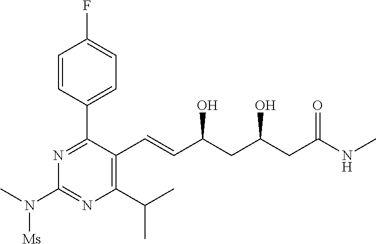

This reaction was performed in the dark. Lactone 1 (45 mg, 0.097 mmol) was dissolved in THF (0.8 mL) and methanamine (33% in EtOH, 0.060 mL, 0.485 mmol) was added. The solution was stirred at rt for 2.5 h. Next, the mixture was concentrated under reduced pressure. Purification by flash chromatography (CH₂Cl₂:MeOH=95:5→85:15) afforded 3 as a white foam (47 mg, 98%). ¹H NMR (400 MHz, CDCl₃) δ 7.72-7.55 (m, 2H), 7.11-7.06 (m, 2H), 6.64 (dd, J=16.1, 1.5 Hz, 1H), 5.70-5.64 (m, 1H), 5.43 (dd, J=16.0, 5.2 Hz, 1H), 4.67 (d, J=1.9 Hz, 1H), 4.49-4.43 (m, 1H), 4.22-4.15 (m, 1H), 3.66-3.64 (br s, 1H), 3.57 (s, 3H), 3.52 (s, 3H), 3.40-3.32 (m, 1H), 2.84 (d, J=4.9 Hz, 3H), 2.31-2.28 (m, 2H), 1.58-1.48 (m, 1H), 1.43-1.37 (m, 1H), 1.26 (dd, J=6.7, 0.9 Hz, 6H).

### (3R,5S,E)-7-(4-(4-fluorophenyl)-6-isopropyl-2-(N-methylmethylsulfonamido)-pyrimidin-5-yl)-3,5-dihydroxy-N,N-dimethylhept-6-enamide 4

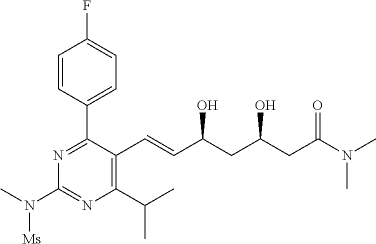

This reaction was performed in the dark. Lactone 1 (65 mg, 0.14 mmol) was dissolved in THF (1.5 mL) and dimethylamine (40% in water, 0.444 mL, 3.51 mmol) was added. The solution was stirred at rt for 5 h. Next, the mixture was concentrated under reduced pressure. Purification by flash chromatography (CH₂Cl₂:MeOH=97:3→90:10) afforded 4 as a white foam (45 mg, 65%). ¹H NMR (400 MHz, CDCl₃) δ 7.69-7.64 (m, 2H), 7.12-7.06 (m, 2H), 6.66 (dd, J=16.1, 1.6 Hz, 1H), 5.45 (dd, J=16.0, 5.0 Hz, 1H), 4.95-4.92 (m, 1H), 4.50-4.44 (m, 1H), 4.27-4.19 (m, 1H), 4.12 (s, 1H), 3.57 (s, 3H), 3.52 (s, 3H), 3.44-3.32 (m, 1H), 2.99 (s, 3H), 2.97 (s, 3H), 2.46-2.29 (m, 2H), 1.61-1.51 (m, 1H), 1.44-1.37 (m, 1H), 1.26 (d, J=6.7 Hz, 6H).

### (3R,5S,E)-7-(4-(4-fluorophenyl)-6-isopropyl-2-(N-methylmethylsulfonamido)-pyrimidin-5-yl)-3,5-dihydroxyhept-6-enamide 5

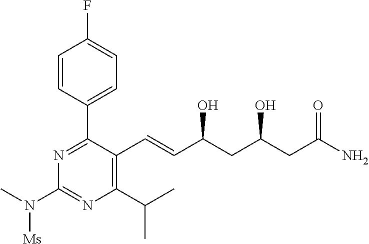 [SMILES: CC(C)c1nc(N(C)S(C)(=O)=O)nc(-c2ccc(F)cc2)c1/C=C/[C@@H](O)C[C@@H](O)CC(N)=O]

This reaction was performed in the dark. Lactone 1 (425 mg, 0.92 mmol) was dissolved in ammonia (7N in MeOH, 6.5 mL, 46 mmol) and the solution was stirred at rt for 16 h. Next, the mixture was concentrated under reduced pressure. Purification by flash chromatography (CH₂Cl₂:MeOH=92.5:7.5→85:15) afforded 5 as a white foam (433 mg, 98%). ¹H NMR (400 MHz, CDCl₃) δ 7.67-7.61 (m, 2H), 7.12-7.06 (m, 2H), 6.64 (dd, J=16.1, 1.6 Hz, 1H), 5.73 (br s, 1H), 5.44 (dd, J=16.1, 5.3 Hz, 1H), 5.42 (br s, 1H), 4.50-4.44 (s, 1H), 4.43-4.40 (m, 1H), 4.26-4.18 (m, 1H), 3.57 (s, 3H), 3.52 (s, 3H), 3.50 (d, J=1.9 Hz, 1H), 3.41-3.30 (m, 1H), 2.39-2.34 (m, 2H), 1.62-1.51 (m, 1H), 1.46-1.40 (m, 1H), 1.26 (dd, J=6.7, 1.2 Hz, 6H).

### 2-((4R,6S)-6-((E)-2-(4-(4-fluorophenyl)-6-isopropyl-2-(N-methylmethyl-sulfonamido)pyrimidin-5-yl)vinyl)-2,2-dimethyl-1,3-dioxan-4-yl)acetamide 6

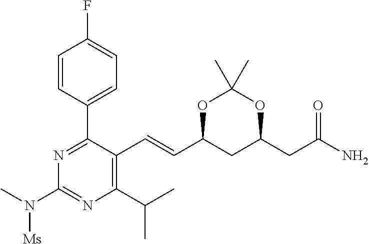 [SMILES: CC(C)c1nc(N(C)S(C)(=O)=O)nc(-c2ccc(F)cc2)c1/C=C/[C@@H]1C[C@H](CC(N)=O)OC(C)(C)O1]

This reaction was performed in the dark. Diol 5 (390 mg, 0.81 mmol) was dissolved in acetone (8 mL) and the solution was cooled in an ice bath. Then, 2-methoxyprop-1-ene (0.23 mL, 2.43 mmol) and p-toluenesulfonic acid monohydrate (7.72 mg, 0.041 mmol) were added. After stirring for 30 min the ice bath was removed. After an additional 1.5 h the mixture was diluted with saturated aqueous NaHCO₃ (25 mL), EtOAc (25 mL) and water (5 mL). The layers were mixed, separated and the aqueous phase was extracted with EtOAc (15 mL). The combined organic layers were washed with brine (10 mL), dried over Na₂SO₄ and filtered before concentration under reduced pressure. Purification by flash chromatography (CH₂Cl₂:MeOH=100:2.5→100:10) afforded 6 as a white foam (405 mg, 96%). ¹H NMR (400 MHz, CDCl₃) δ 7.67-7.61 (m, 2H), 7.12-7.05 (m, 2H), 6.52 (dd, J=16.2, 1.4 Hz, 1H), 6.10 (br s, 1H), 5.46 (dd, J=16.2, 5.4 Hz, 1H), 5.33 (br s, 1H), 4.48-4.41 (m, 1H), 4.33-4.24 (m, 1H), 3.57 (s, 3H), 3.52 (s, 3H), 3.42-3.31 (m, 1H), 2.44 (ABdd, J=15.1, 7.4 Hz, 1H), 2.35 (ABdd, J=15.2, 4.2 Hz, 1H), 1.56-1.52 (m, 1H), 1.50 (s, 3H), 1.43 (s, 3H), 1.27 (dd, J=6.7, 3.2 Hz, 6H), 1.26-1.14 (m, 1H).

### N-(5-((E)-2-((4S,6S)-6-(cyanomethyl)-2,2-dimethyl-1,3-dioxan-4-yl)vinyl)-4-(4-fluorophenyl)-6-isopropylpyrimidin-2-yl)-N-methylmethanesulfonamide 7

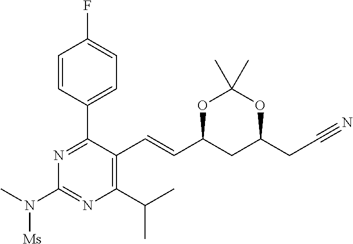 [SMILES: CC(C)c1nc(N(C)S(C)(=O)=O)nc(-c2ccc(F)cc2)c1/C=C/[C@@H]1C[C@H](CC#N)OC(C)(C)O1]

This reaction was performed in the dark. Dioxolane 6 (405 mg, 0.77 mmol) was dissolved in dry EtOAc (8 mL) and the solution was cooled with an ice bath. Then, dry DMF (205 μL, 2.64 mmol) and Et₃N (347 μL, 2.49 mmol) were added, followed by the slow addition of phosphoryl trichloride (218 μL, 2.33 mmol) over a period of 10 min. After stirring the mixture for 45 min in the ice bath, the yellow mixture was diluted with saturated aqueous NaHCO₃ (15 mL) and EtOAC (15 mL) and allowed to warm to rt. The layers were mixed, separated and the aqueous phase was extracted with EtOAc (10 mL). The combined organic layers were washed with saturated aqueous NaHCO₃ (15 mL) and brine (2×15 mL), dried over Na₂SO₄ and filtered before concentration under reduced pressure. Purification by flash chromatography (heptane:EtOAc=2.5:1→1:1) afforded 7 as a white foam (338 mg, 86%). ¹H NMR (400 MHz, CDCl₃) δ 7.68-7.62 (m, 2H), 7.14-7.07 (m, 2H), 6.55 (dd, J=16.2, 1.5 Hz, 1H), 5.47 (dd, J=16.2, 5.3 Hz, 1H), 4.46-4.40 (m, 1H), 4.20-4.10 (m, 1H), 3.57 (s, 3H), 3.52 (s, 3H), 3.42-3.30 (m, 1H), 2.55 (ABdd, J=16.7, 5.8 Hz, 1H), 2.49 (ABdd, J=16.7, 6.2 Hz, 1H), 1.67-1.60 (m, 1H), 1.48 (s, 3H), 1.44 (s, 3H), 1.28 (dd, J=6.7, 1.9 Hz, 6H), 1.27-1.20 (m, 1H).

### N-(5-((3S,5S,E)-6-cyano-3,5-dihydroxyhex-1-en-1-yl)-4-(4-fluorophenyl)-6-isopropylpyrimidin-2-yl)-N-methylmethanesulfonamide 8

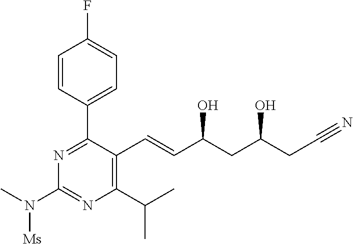 [SMILES: CC(C)c1nc(N(C)S(C)(=O)=O)nc(-c2ccc(F)cc2)c1/C=C/[C@@H](O)C[C@@H](O)CC#N]

This reaction was performed in the dark. Nitrile 7 (50 mg, 99 gmol) was dissolved in MeCN (0.5 mL) and 0.2M aqueous HCl (497 μL, 99 gmol) and the mixture was stirred at rt for 3 h. Then, the mixture was diluted with EtOAc (5 mL) and saturated aqueous NaHCO₃ (5 mL). The layers were mixed, separated and the aqueous phase was extracted with EtOAc (2×5 mL). The combined organic layers were washed with brine (5 mL), dried over Na₂SO₄ and filtered before concentration under reduced pressure. After purification by flash chromatography (heptane:EtOAc=1:2→1:10) compound 8 was isolated as a colorless oil (38 mg, 83%). ¹H NMR (400 MHz, CDCl₃) δ 7.64-7.57 (m, 2H), 7.15-7.09 (m, 2H), 6.63 (dd, J=16.1, 1.4 Hz, 1H), 5.48 (dd, J=16.1, 6.0 Hz, 1H), 4.53-4.45 (m, 1H), 4.18-4.12 (m, 1H), 3.66-3.62 (m, 1H), 3.57 (s, 3H), 3.52 (s, 3H), 3.36-3.25 (m, 1H), 2.60-2.47 (m, 2H), 2.43 (d, J=3.1 Hz, 1H), 1.71-1.63 (m, 2H), 1.28 (d, J=6.7 Hz, 6H).

### N-(5-((E)-2-((4S,6S)-6-((1H-tetrazol-5-yl)methyl)-2,2-dimethyl-1,3-dioxan-4-yl)vinyl)-4-(4-fluorophenyl)-6-isopropylpyrimidin-2-yl)-N-methylmethane sulfonamide 9

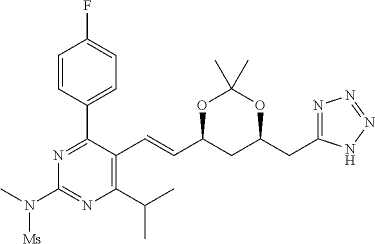 [SMILES: CC(C)c1nc(N(C)S(C)(=O)=O)nc(-c2ccc(F)cc2)c1/C=C/[C@@H]1C[C@H](Cc2nnn[nH]2)OC(C)(C)O1]

This reaction was performed in the dark. Nitrile 7 (125 mg, 0.25 mmol) was dissolved in DMF (1 mL) and NH₄Cl (133 mg, 2.49 mmol) and NaN₃ (162 mg, 2.49 mmol) were added. The mixture was stirred for 5 min at rt, then warmed to 115° C. and stirred for an additional 20 h. The mixture was cooled to rt and diluted with EtOAc (30 mL) and water (20 mL). Under vigorous stirring, the mixture was acidified to pH=2.5 using 1M aqueous HCl. The layers were separated and the aqueous phase was extracted with EtOAc (10 mL). The combined organic layers were washed with brine (3×15 mL), dried over Na₂SO₄ and filtered before concentration under reduced pressure. Purification by flash chromatography (CH₂Cl₂:MeOH=100:2→100:6) afforded 9 as a colorless oil (65 mg, 83%, purity ˜87% based on HPLC).

### N-(5-((3S,5S,E)-3,5-dihydroxy-6-(1H-tetrazol-5-yl)hex-1-en-1-yl)-4-(4-fluorophenyl)-6-isopropylpyrimidin-2-yl)-N-methylmethanesulfonamide 10

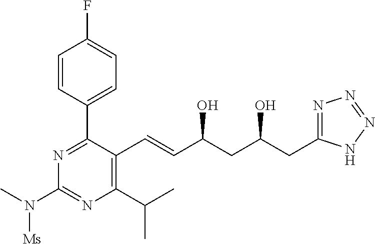 [SMILES: CC(C)c1nc(N(C)S(C)(=O)=O)nc(-c2ccc(F)cc2)c1/C=C/[C@@H](O)C[C@@H](O)Cc1nnn[nH]1]

This reaction was performed in the dark. Compound 9 (65 mg, 0.12 mmol) was dissolved in MeCN (0.6 mL) and 0.2M aqueous HCl (596 μL, 0.12 mmol) and the mixture was stirred at rt for 3.5 h. The mixture was diluted with EtOAc (10 mL) and saturated aqueous NH₄Cl (10 mL). The layers were mixed, separated and the aqueous phase was extracted with EtOAc (5 mL). The combined organic layers were washed with brine (5 mL), dried over Na₂SO₄ and filtered before concentration under reduced pressure. Purification by flash chromatography (CH₂Cl₂:MeOH=100:5→100:15) afforded 10 as a white foam (40 mg, 66%). ¹H NMR (400 MHz, CDCl₃) δ 7.61-7.54 (m, 2H), 7.11-7.03 (m, 2H), 6.60 (dd, J=16.1, 1.2 Hz, 1H), 5.47 (dd, J=16.1, 6.1 Hz, 1H), 4.55-4.48 (m, 1H), 4.31-4.23 (m, 1H), 3.57 (s, 3H), 3.51 (s, 3H), 3.34-3.18 (m, 2H), 3.05 (ABdd, J=15.5, 7.9 Hz, 1H), 1.69-1.44 (m, 2H), 1.25 (dd, J=6.7, 0.9 Hz, 6H).

### N-(5-((E)-2-((4S,6S)-2,2-dimethyl-6-((5-oxo-2,5-dihydro-1,2,4-oxadiazol-3-yl)methyl)-1,3-dioxan-4-yl)vinyl)-4-(4-fluorophenyl)-6-isopropylpyrimidin-2-yl)-N-methylmethanesulfonamide 11

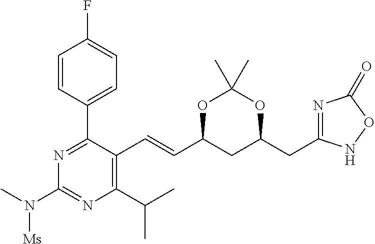 [SMILES: CC(C)c1nc(N(C)S(C)(=O)=O)nc(-c2ccc(F)cc2)c1/C=C/[C@@H]1C[C@H](Cc2nc(=O)o[nH]2)OC(C)(C)O1]

This reaction was performed in the dark. Nitrile 7 (100 mg, 0.12 mmol) was dissolved in MeOH (1 mL) and aqueous hydroxylamine (50% in water, 197 μL, 2.98 mmol) was added. The mixture was stirred for 5 min at rt and then warmed to 65° C. After stirring for another 4.5 h, the mixture was allowed to cool to rt, diluted with THF and concentrated under reduced pressure. The residue was stripped with THF once more. The remaining oil was dissolved in THF (1 mL) and CDI (48.4 mg, 0.30 mmol) was added, followed after 10 min by Diazabicycloundecene (2,3,4,6,7,8,9,10-octahydropyrimido[1,2-a]azepine; DBU; 37 μL, 0.25 mmol). The mixture was stirred at rt for 16 h. Then, more CDI (60 mg) was added, followed by DBU (30 μL, after 10 min) and after stirring for 1 h again more CDI (40 mg) and DBU (20 μL) were added. After an additional 1 h, the mixture was diluted with saturated aqueous NH₄Cl (15 mL) and EtOAc (15 mL). The layers were mixed, separated and the organic phase was washed with brine (10 mL), dried over Na₂SO₄ and filtered before concentration under reduced pressure. Purification by flash chromatography (heptane:EtOAc=1:1→1:3) afforded 11 as a white foam (103 mg, 92%).

### N-(5-((3S,5S,E)-3,5-dihydroxy-6-(5-oxo-2,5-dihydro-1,2,4-oxadiazol-3-yl)hex-1-en-1-yl)-4-(4-fluorophenyl)-6-isopropylpyrimidin-2-yl)-N-methylmethane Sulfonamide 12

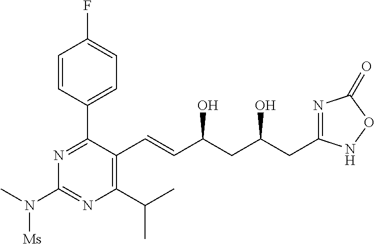 [SMILES: CC(C)c1nc(N(C)S(C)(=O)=O)nc(-c2ccc(F)cc2)c1/C=C/[C@@H](O)C[C@@H](O)Cc1nc(=O)o[nH]1]

This reaction was performed in the dark. Compound 11 (100 mg, 0.18 mmol) was dissolved in MeCN (0.9 mL) and 0.2 M aqueous HCl (890 μL, 0.18 mmol) and the mixture was stirred at rt for 3 h. Next, the mixture was diluted with EtOAc (10 mL) and saturated aqueous NH₄Cl (10 mL). The layers were mixed, separated and the aqueous phase was extracted with EtOAc (5 mL). The combined organic layers were washed with brine (15 mL), dried over Na₂SO₄ and filtered before concentration under reduced pressure. Purification by flash chromatography (CH₂Cl₂:MeOH=100:2.5→100:10) afforded 12 as a white foam (60 mg, 64%). ¹H NMR (400 MHz, CDCl₃) δ 7.55-7.62 (m, 2H), 7.13-7.06 (m, 2H), 6.60 (dd, J=16.1, 1.0 Hz, 1H), 5.46 (dd, J=16.1, 6.1 Hz, 1H), 4.54-4.46 (m, 1H), 4.26-4.18 (m, 1H), 3.57 (s, 3H), 3.51 (s, 3H), 3.35-3.22 (m, 1H), 2.78 (ABdd, J=15.2, 3.3 Hz, 1H), 2.63 (ABdd, J=15.2, 7.6 Hz, 1H), 1.60-1.53 (m, 2H), 1.26 (d, J=6.7 Hz, 6H).

### (S)-4-benzyl-3-(2-(benzyloxy)acetyl)oxazolidin-2-one 13

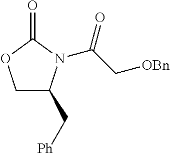 [SMILES: O=C(COCc1ccccc1)N1C(=O)OC[C@@H]1Cc1ccccc1]

To a water bath-cooled suspension of (S)-4-benzyloxazolidin-2-one (4.44 g, 25.1 mmol) in dry toluene (45 mL) were added subsequently, benzyloxyacetic acid (5.0 g, 30.1 mmol) and Et₃N (8.74 mL, 62.7 mmol). The mixture became clear and was stirred for 5 min. Then, pivaloyl chloride (3.70 mL, 30.1 mmol) was added, followed by DMAP (1.53 g, 12.5 mmol), and the thick mixture was stirred for 5 min at rt, followed by stirring at 75° C. for 16 h. The mixture was cooled to rt, after which the reaction mixture was washed twice with 1M aqueous HCl (50 mL, then 25 mL), 0.5M aqueous NaOH (3×25 mL) and brine (25 mL), dried over Na₂SO₄ and filtered before concentration under reduced pressure. Purification by flash chromatography (heptane:EtOAc=3:1→1.5:1) afforded 13 as a white solid (5.75 g, 70%). ¹H NMR (400 MHz, CDCl₃) δ 7.45-7.27 (m, 8H), 7.23-7.18 (m, 2H), 4.74-4.66 (m, 5H), 4.31-4.20 (m, 2H), 3.33 (ABdd, J=13.4, 3.3 Hz, 1H), 2.82 (ABdd, J=13.4, 9.4 Hz, 1H).

### (S)-4-benzyl-3-((R)-2-(benzyloxy)pent-4-enoyl)oxazolidin-2-one 14

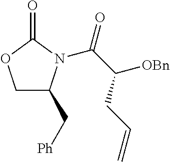 [SMILES: C=CC[C@@H](OCc1ccccc1)C(=O)N1C(=O)OC[C@@H]1Cc1ccccc1]

A solution of 13 (6.87 g, 21.1 mmol) in THF (106 mL) was cooled to −75° C. and sodium bis(trimethylsilyl)amide (2M in THF, 15.8 mL, 31.7 mmol) was added over a period of 5 min. The resulting yellow mixture was stirred for 25 min during which the temperature changed from −75° C. to −45° C. The mixture was cooled again to −70° C. and a solution of 3-iodoprop-1-ene (5.78 mL, 63.3 mmol) in THF (15 mL) was added dropwise over a period of 5 min. The mixture was allowed to warm to −35° C. over a period of 1 h. After stirring for an additional 1 h at −35° C., the reaction was quenched by the addition of saturated aqueous NH₄Cl (250 mL). The mixture was diluted with EtOAc (250 mL) and water (25 mL), the layers were mixed and separated. The aqueous phase was extracted with EtOAc (100 mL) and the combined organic layers were washed with saturated aqueous NH₄Cl (250 mL), saturated aqueous NaHCO₃ (200 mL) and brine (200 mL), dried over Na₂SO₄ and filtered before concentration under reduced pressure. Purification by flash chromatography (heptane:EtOAc=5:1→3:1) afforded 14 as a yellow oil (4.63 g, 60%, purity ˜94% based on HPLC).

### (3R,5S)-3-(benzyloxy)-5-(hydroxymethyl)dihydrofuran-2(3H)-one 15

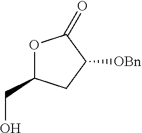 [SMILES: O=C1O[C@H](CO)C[C@H]1OCc1ccccc1]

A solution of 14 (4.6 g, 12.6 mmol) in THF (100 mL) and water (16 mL) was cooled in an ice bath and N-Methylmorpholine N-oxide (NMO; 10.3 g, 88 mmol) was added, followed by a 4% aqueous solution of OsO₄ (3.1 mL, 0.50 mmol). The mixture was stirred at rt for 17 h. The solution was poured into a saturated aqueous Na₂S₂O₃ solution (250 mL) and the mixture was stirred for 10 min, before EtOAc (300 mL) was added together with brine (100 mL). The layers were mixed, separated and the aqueous phase was extracted with EtOAc (75 mL). The combined organic layers were washed with brine (150 mL), dried over Na₂SO₄ and filtered before concentration under reduced pressure. Purification by flash chromatography (heptane:EtOAc=1.5:1→1:2) afforded 15 as a yellow oil (1.8 g) contaminated with (S)-4-benzyloxazolidin-2-one (Evans chiral auxiliary). The theoretical yield was 50% (6.3 mmol). ¹H NMR (400 MHz, CDCl₃) δ 7.39-7.28 (m, 5H), 4.93 (d, J=11.6 Hz, 1H), 4.74-4.68 (m, 1H), 4.68 (d, J=11.6 Hz, 1H), 4.36-4.31 (m, 1H), 3.95-3.88 (m, 1H), 3.59 (ddd, J=12.5, 6.4, 3.6 Hz, 1H), 2.46-2.39 (m, 1H), 2.33-2.25 (m, 2H).

### (3R,5S)-5-(((tert-butyldimethylsilyl)oxy)methyl)-3-hydroxydihydrofuran-2(3H)-one 16

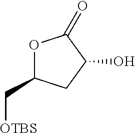 [SMILES: CC(C)(C)[Si](C)(C)OC[C@@H]1C[C@@H](O)C(=O)O1]

A solution of 15 (6.3 mmol) in CH₂Cl₂ (60 mL) was cooled in an ice bath and imidazole (1.6 g, 24 mmol) and TBS-Cl (1.4 g, 9.3 mmol) were added. The mixture was stirred at rt for 3 h. The solution was quenched by the addition of saturated aqueous NH₄Cl (150 mL) and diluted with EtOAc (120 mL). The layers were mixed, separated and the organic phase was washed with saturated aqueous NH₄Cl (75 mL), saturated aqueous NaHCO₃ (75 mL) and brine (75 mL), dried over Na₂SO₄ and filtered before concentration under reduced pressure. Purification by flash chromatography (heptane:EtOAc=12:1→5:1) afforded 16 as a colorless oil (1.72 g, 81%). ¹H NMR (400 MHz, CDCl₃) δ 7.40-7.29 (m, 5H), 4.94 (d, J=11.9 Hz, 1H), 4.74 (d, J=11.9 Hz, 1H), 4.65-4.60 (m, 1H), 4.36 (dd, J=8.5, 7.8 Hz, 1H), 3.85 (ABdd, J=11.4, 2.5 Hz, 1H), 3.60 (ABdd, J=11.4, 2.2 Hz, 1H), 2.42 (ddd, J=13.1, 8.5, 3.0 Hz, 1H), 2.33-2.25 (m, 1H), 0.82 (s, 9H), 0.02 (s, 6H). The obtained material was dissolved in EtOH (35 mL) and Pd/C (Degussa-type, 435 mg, 0.20 mmol) was added. The suspension was saturated with H₂ gas, while stirring vigorously for 3 h. The suspension was filtered through Celite®, which was rinsed with EtOH (2×25 mL). The filtrate was concentrated under reduced pressure and stripped with CH₂Cl₂ to yield 16 (1.20 g, 95%). ¹H NMR (400 MHz, CDCl₃) δ 4.70-4.63 (m, 2H), 3.89 (ABdd, J=11.4, 2.4 Hz, 1H), 3.65 (ABdd, J=11.4, 2.1 Hz, 1H), 2.69 (br s, 1H), 2.58 (ddd, J=12.9, 9.0, 1.8 Hz, 1H), 2.31 (dt, J=12.9, 9.0 Hz, 1H), 0.88 (s, 9H), 0.07 (s, 3H), 0.06 (s, 3H).

### Methyl (4R,6S)-6-(((tert-butyldimethylsilyl)oxy)methyl)-2,2-dimethyl-1,3-dioxane-4-carboxylate 17

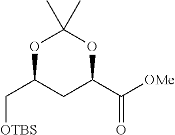 [SMILES: COC(=O)[C@H]1C[C@@H](CO[Si](C)(C)C(C)(C)C)OC(C)(C)O1]

A solution of 16 (1.0 g, 4.1 mmol) in dioxane (30 mL) and water (20 mL) was cooled in an ice bath and 1M aqueous LiOH (4.14 mL, 4.14 mmol) was added. The mixture was stirred for 50 min, after which more 1M aqueous LiOH (200 jtL) was added. After another 50 min again more 1M aqueous LiOH (250 μL) was added. After another 2.5 h, the reaction was quenched by the slow addition of 1M aqueous HCl (4.46 mL, 4.46 mmol) and the pH was checked to be ˜4.5. The mixture was lyophilized to yield an oil (1.4 g). Of this oil, 0.9 g (˜3 mmol) was suspended in Et₂O (40 mL), cooled in an ice bath and stirred for 5 min. The mixture was then filtered through Celite®, which was rinsed twice with Et₂O (10 and 5 mL). The obtained clear solution was cooled in an ice bath and diazomethane was added from a stock solution in Et₂O using a plastic syringe until a yellow color persisted. The mixture was stirred for an additional 5 min and the excess diazomethane was removed by bubbling through N₂ gas for 10 min at 4° C. The colorless solution was diluted with acetone (40 mL) and 2-methoxyprop-1-ene (1.66 mL, 17.2 mmol) was added, followed by camphorsulfonic acid (CSA; 0.10 g, 0.43 mmol) in 4 portions over 8 min. After 20 min, more CSA (25 mg) and 2-methoxyprop-1-ene (0.5 mL) were added. After stirring for another 20 min, the mixture was diluted with saturated aqueous NaHCO₃ (100 mL) and water (10 mL) and extracted twice with EtOAc (2×70 mL) and the combined organic layers were washed with saturated aqueous NaHCO₃ (50 mL) and brine (50 mL), dried over Na₂SO₄ and filtered before concentration under reduced pressure. Purification by flash chromatography (heptane:EtOAc=8:1→3:1) afforded 17 as a colorless oil (0.36 g, ˜35-40% yield). ¹H NMR (400 MHz, CDCl₃) δ 4.53 (dd, J=12.2, 2.8 Hz, 1H), 4.01-3.95 (m, 1H), 3.78 (s, 3H), 3.68 (ABdd, J=10.3, 5.2 Hz, 1H), 3.50 (ABdd, J=10.3, 6.0 Hz, 1H), 1.94 (dt, J=13.0, 2.7 Hz, 1H), 1.48 (s, 3H), 1.48 (s, 3H), 0.89 (s, 9H), 0.06 (s, 3H), 0.06 (s, 3H).

### Methyl (4R,6S)-6-(hydroxymethyl)-2,2-dimethyl-1,3-dioxane-4-carboxylate 18

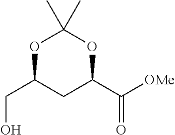

A solution of 17 (398 mg, 1.25 mmol) in THF (6 mL) was cooled in an ice bath and TBAF (1M in THF, 1.31 mL, 1.31 mmol) was added. The mixture was stirred for 1 h, after which more TBAF (100 μL) was added. After another 1.5 h again more TBAF (75 μL) was added. After another 2 h, the reaction was quenched by the addition of saturated aqueous NH₄Cl (25 mL) and EtOAc (20 mL). The layers were mixed, separated and the aqueous phase was extracted with EtOAc (3×15 mL). The combined organic layers were dried over Na₂SO₄ and filtered before concentration under reduced pressure. Purification by flash chromatography (heptane:EtOAc=1:1→1:4) afforded 18 as a colorless oil (0.17 g, 66%). ¹H NMR (400 MHz, CDCl₃) δ 4.55 (dd, J=12.1, 3.0 Hz, 1H), 4.10-4.04 (m, 1H), 3.77 (s, 3H), 3.65 (br s, 1H), 3.54 (ABdd, J=11.4, 5.9 Hz, 1H), 2.12 (br s, 1H), 1.79 (dt, J=13.0, 2.9 Hz, 1H), 1.71-1.63 (m, 1H), 1.51 (s, 3H), 1.50 (s, 3H).

### Methyl (4R,6S)-6-formyl-2,2-dimethyl-1,3-dioxane-4-carboxylate 19

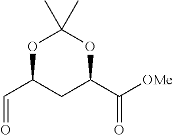

A solution of 18 (170 mg, 0.832 mmol) in CH₂Cl₂ (5.5 mL) was cooled in an ice bath and Dess-Martin periodinane (424 mg, 0.99 mmol) was added in 4 portions over 5 min. The mixture was stirred at rt for 1.5 h, after which more Dess-Martin periodinane (50 mg) was added. After stirring for another 5 min, the reaction was quenched by the addition of a mixture of saturated aqueous NaHCO₃ (15 mL) and Na₂S₂O₃ (4.5 g). After stirring for 2 min the mixture was diluted with CH₂Cl₂ (10 mL). The layers were mixed, separated and the aqueous phase was extracted with CH₂Cl₂ (3×10 mL). The combined organic layers were dried over Na₂SO₄ and filtered before concentration under reduced pressure. Purification by flash chromatography (heptane:EtOAc=1:1→1:3) afforded 19 as a colorless oil (0.11 g, 65%). ¹H NMR (400 MHz, CDCl₃) δ 9.59 (s, 1H), 4.56 (dd, J=12.1, 2.8 Hz, 1H), 4.35 (dd, J=12.1, 3.1 Hz, 1H), 3.78 (s, 3H), 2.11 (dt, J=13.2, 2.9 Hz, 1H), 1.73-1.60 (m, 1H), 1.58 (s, 3H), 1.52 (s, 3H).

### Methyl (4R,6S)-6-((E)-2-(4-(4-fluorophenyl)-6-isopropyl-2-(N-methylmethyl-sulfonamido)pyrimidin-5-yl)vinyl)-2,2-dimethyl-1,3-dioxane-4-carboxylate 21

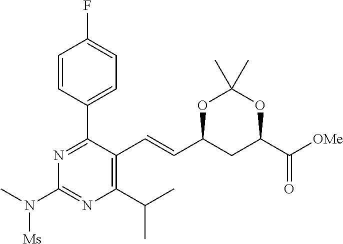 [SMILES: COC(=O)[C@H]1C[C@@H](/C=C/c2c(-c3ccc(F)cc3)nc(N(C)S(C)(=O)=O)nc2C(C)C)OC(C)(C)O1]

This reaction was performed in the dark. To a cooled (4° C.) solution of 19 (369 mg, 0.54 mmol) in DMSO (3 mL) was added a solution of 20 (369 mg, 0.54 mmol) (Org. Biomol. Chem., 2016, 14, 1363-1369) in DMSO (2 m), followed by K₂C₃ (90 mg, 0.65 mmol). DMSO (2 mL), followed by K₂CO₃ (90 mg, 0.65 mmol). The mixture was warmed to 65° C. and stirred for 1.5 h. Next, the mixture was diluted with toluene (35 mL) and saturated aqueous NH₄Cl (25 mL). The layers were mixed, separated and the organic layer was washed with brine (2×10 mL), dried over Na₂SO₄ and filtered before concentration under reduced pressure. Purification by flash chromatography (heptane:EtOAc=3:1→1:1) afforded 21 as a colorless oil (0.16 g, 57%). ¹H NMR (400 MHz, CDCl₃) δ 7.67-7.60 (m, 2H), 7.13-7.05 (m, 2H), 6.56 (dd, J=16.2, 1.5 Hz, 1H), 5.47 (dd, J=16.2, 5.2 Hz, 1H), 4.54 (dd, J=12.1, 2.7 Hz, 1H), 4.51-4.43 (m, 1H), 3.79 (s, 3H), 3.57 (s, 3H), 3.52 (s, 3H), 3.36 (hept, J=6.7 Hz, 1H), 1.81 (dt, J=13.0, 2.7 Hz, 1H), 1.53 (s, 3H), 1.51 (s, 3H), 1.52-1.41 (m, 1H), 1.27 (dd, J=6.7, 3.7 Hz, 6H).

### (4R,6S)-6-((E)-2-(4-(4-fluorophenyl)-6-isopropyl-2-(N-methylmethyl sulfonamido)pyrimidin-5-yl)vinyl)-2,2-dimethyl-1,3-dioxane-4-carboxamide 22

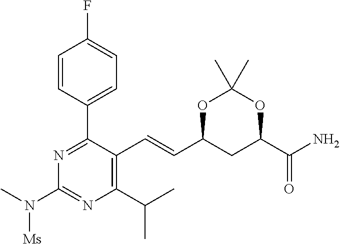 [SMILES: CC(C)c1nc(N(C)S(C)(=O)=O)nc(-c2ccc(F)cc2)c1/C=C/[C@@H]1C[C@H](C(N)=O)OC(C)(C)O1]

Compound 21 (164 mg, 0.314 mmol) was dissolved in ammonia (7N in MeOH, 3.37 ml, 23.6 mmol) and the solution was stirred in the dark for 2.5 h. The mixture was concentrated and after purification by flash chromatography (heptane:EtOAc=1:2→1:4) compound 22 was isolated as a colorless oil (143 mg, 90%). ¹H NMR (400 MHz, CDCl₃) δ 7.69-7.61 (m, 2H), 7.12-7.06 (m, 2H), 6.54 (dd, J=16.3, 1.4 Hz, 1H), 6.50 (br s, 1H), 5.73 (br s, 1H), 5.50 (dd, J=16.3, 5.4 Hz, 1H), 4.52-4.45 (m, 1H), 4.37 (dd, J=12.1, 2.8 Hz, 1H), 3.57 (s, 3H), 3.51 (s, 3H), 3.36 (hept, J=6.7 Hz, 1H), 2.02 (dt, J=13.1, 2.6 Hz, 1H), 1.50 (s, 3H), 1.48 (s, 3H), 1.39-1.29 (m, 1H), 1.27 (dd, J=6.7, 3.6 Hz, 6H).

### N-(5-((E)-2-((4S,6R)-6-cyano-2,2-dimethyl-1,3-dioxan-4-yl)vinyl)-4-(4-fluorophenyl)-6-isopropylpyrimidin-2-yl)-N-methylmethanesulfonamide 23

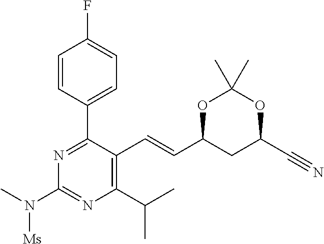 [SMILES: CC(C)c1nc(N(C)S(C)(=O)=O)nc(-c2ccc(F)cc2)c1/C=C/[C@@H]1C[C@H](C#N)OC(C)(C)O1]

This reaction was performed in the dark. Amide 22 (116 mg, 0.23 mmol) was dissolved in dry CH₂Cl₂ (5 mL) and after the addition of dry DMSO (146 μL, 2.06 mmol) the solution was cooled to −70° C. Then, oxalyl chloride (80 μL, 0.92 mmol) was added slowly and after 20 min at −70° C., Et₃N (383 μL, 2.75 mmol) was added and the mixture was allowed to warm to −30° C. over a period of 40 min. After stirring at −30° C. for an additional 50 min, the reaction was diluted with saturated aqueous NH₄Cl (25 mL) and EtOAc (20 mL). The layers were mixed, separated and the aqueous phase was extracted with EtOAc (10 mL). The combined organic layers were washed with brine (15 mL), dried over Na₂SO₄ and filtered before concentration under reduced pressure. Purification by flash chromatography (heptane:EtOAc=3:1→1:1) afforded 23 as a colorless oil (104 mg, 93%). ¹H NMR (400 MHz, CDCl₃) δ 7.66-7.61 (m, 2H), 7.14-7.08 (m, 2H), 6.59 (dd, J=16.2, 1.5 Hz, 1H), 5.45 (dd, J=16.2, 5.2 Hz, 1H), 4.80-4.77 (m, 1H), 4.44-4.39 (m, 1H), 3.57 (s, 3H), 3.52 (s, 3H), 3.33 (hept, J=6.8 Hz, 1H), 1.82-1.71 (m, 2H), 1.49 (s, 3H), 1.48 (s, 3H), 1.28 (dd, J=6.7, 2.2 Hz, 6H).

### N-(5-((E)-2-((4S,6R)-2,2-dimethyl-6-(1H-tetrazol-5-yl)-1,3-dioxan-4-yl)vinyl)-4-(4-fluorophenyl)-6-isopropylpyrimidin-2-yl)-N-methylmethanesulfonamide 24

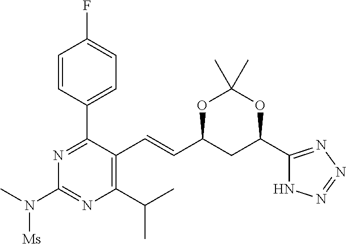 [SMILES: CC(C)c1nc(N(C)S(C)(=O)=O)nc(-c2ccc(F)cc2)c1/C=C/[C@@H]1C[C@H](c2nnn[nH]2)OC(C)(C)O1]

This reaction was performed in the dark. Nitrile 23 (35 mg, 72 μmol) was dissolved in DMF (0.3 mL) and ammonium chloride (77 mg, 1.43 mmol) and sodium azide (93 mg, 1.43 mmol) were added. The mixture was stirred for 5 min at rt, then warmed to 115° C. and stirred for another 1.5 h. The reaction mixture was allowed to cool to rt, and diluted with EtOAc (10 mL) and water (7 mL). The mixture was acidified to pH=4.0 using 1M aqueous HCl. The layers were mixed, separated and the aqueous phase was extracted with EtOAc (10 mL). The combined organic layers were washed with brine (2×6 mL), dried over Na₂SO₄ and filtered before concentration under reduced pressure. Purification by flash chromatography (CH₂Cl₂:MeOH=100:4→100:12) afforded 24 as a colorless oil (30 mg, 80%). ¹H NMR (400 MHz, CDCl₃) δ 7.66-7.61 (m, 2H), 7.11-7.04 (m, 2H), 6.59 (dd, J=16.3, 1.4 Hz, 1H), 5.54-5.44 (m, 2H), 4.66-4.59 (m, 1H), 3.57 (s, 3H), 3.51 (s, 3H), 3.40-3.29 (m, 1H), 2.20-2.13 (m, 1H), 1.61 (s, 3H), 1.60-1.54 (m, 1H), 1.53 (s, 3H), 1.27 (dd, J=6.7, 4.1 Hz, 6H).

### N-(5-((3S,5R,E)-3,5-dihydroxy-5-(1H-tetrazol-5-yl)pent-1-en-1-yl)-4-(4-fluorophenyl)-6-isopropylpyrimidin-2-yl)-N-methylmethanesulfonamide 25

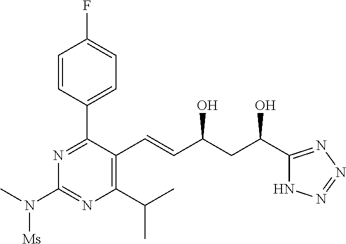 [SMILES: CC(C)c1nc(N(C)S(C)(=O)=O)nc(-c2ccc(F)cc2)c1/C=C/[C@@H](O)C[C@@H](O)c1nnn[nH]1]

This reaction was performed in the dark. Tetrazole 24 (44 mg, 83 μmol) was dissolved in MeCN (0.5 mL) and 0.2M aqueous HCl (0.47 mL, 95 μmol) and the mixture was stirred at rt for 3.5 h. More 0.2M aqueous HCl (50 μL) was added and the mixture was stirred for an additional 1 h. Then, the mixture was diluted with EtOAc (10 mL) and saturated aqueous NH₄Cl (10 mL). The layers were mixed, separated and the organic layer was washed with brine (2×5 mL), dried over Na₂SO₄ and filtered before concentration under reduced pressure. Purification by flash chromatography (CH₂Cl₂:MeOH=100:20→100:50) afforded 25 as a white solid (33 mg, 81%). ¹H NMR (400 MHz, CDCl₃/CD₃OD) δ 7.68-7.61 (m, 2H), 7.12-7.05 (m, 2H), 6.62 (dd, J=16.1, 1.4 Hz, 1H), 5.53 (dd, J=16.1, 5.6 Hz, 1H), 5.22 (dd, J=8.1, 5.3 Hz, 1H), 4.48-4.41 (m, 1H), 3.57 (s, 3H), 3.53 (s, 3H), 3.41-3.34 (m, 1H, partially obscured by CD₃OD residual signal), 2.05-1.88 (m, 2H), 1.26 (dd, J=6.7, 2.0 Hz, 6H).

### N-(5-((E)-2-((4S,6R)-2,2-dimethyl-6-(5-oxo-2,5-dihydro-1,2,4-oxadiazol-3-yl)-1,3-dioxan-4-yl)vinyl)-4-(4-fluorophenyl)-6-isopropylpyrimidin-2-yl)-N-methylmethanesulfonamide 26

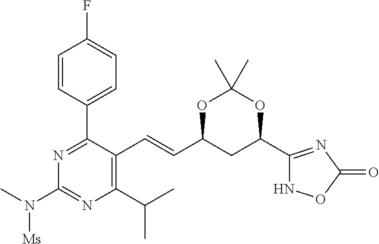 [SMILES: CC(C)c1nc(N(C)S(C)(=O)=O)nc(-c2ccc(F)cc2)c1/C=C/[C@@H]1C[C@H](c2nc(=O)o[nH]2)OC(C)(C)O1]

This reaction was performed in the dark. Nitrile 23 (50 mg, 0.10 mmol) was dissolved in MeOH (0.6 mL) and hydroxylamine (50% solution in water, 0.10 mL, 1.5 mmol) was added. The mixture was stirred for 5 min at rt and then warmed to 65° C. and stirred for an additional 1.5 h. Next, the mixture was allowed to cool to rt, diluted with THF and concentrated under reduced pressure. The residue was stripped with THF once more. The remaining material was dissolved in THF (0.8 mL) and CDI (45 mg, 0.28 mmol) was added, followed after 2 min by DBU (33 μL, 0.22 mmol). The mixture was stirred at rt for 1 h, after which the same amounts of CDI and DBU were added again. After stirring for another 30 min, again the same amounts of CDI and DBU were added once more. After stirring for another 1 h, the mixture was diluted with saturated aqueous NH₄Cl (15 mL) and EtOAc (15 mL). The layers were mixed, separated and the aqueous phase was extracted with EtOAc (10 mL). The combined organic layers were washed with NH₄Cl (10 mL) and with brine (10 mL), dried over Na₂SO₄ and filtered before concentration under reduced pressure. Purification by flash chromatography (CH₂Cl₂:MeOH=100:2.5→100:8) afforded 26 as a colorless oil (44 mg, 81%). ¹H NMR (400 MHz, CDCl₃) δ 7.66-7.61 (m, 2H), 7.13-7.06 (m, 2H), 6.59 (dd, J=16.2, 1.4 Hz, 1H), 5.48 (dd, J=16.2, 5.3 Hz, 1H), 5.02 (dd, J=11.9, 2.8 Hz, 1H), 4.57-4.50 (m, 1H), 3.57 (s, 3H), 3.52 (s, 3H), 3.41-3.28 (m, 1H), 1.88 (dt, J=13.0, 2.7 Hz, 1H), 1.64-1.54 (m, 1H), 1.55 (s, 3H), 1.49 (s, 3H), 1.28 (dd, J=6.7, 3.4 Hz, 6H).

### N-(5-((3S,5R,E)-3,5-dihydroxy-5-(5-oxo-2,5-dihydro-1,2,4-oxadiazol-3-yl)pent-1-en-1-yl)-4-(4-fluorophenyl)-6-isopropylpyrimidin-2-yl)-N-methylmethane-sulfonamide 27

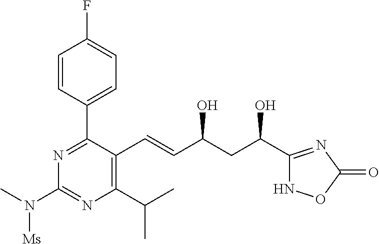 [SMILES: CC(C)c1nc(N(C)S(C)(=O)=O)nc(-c2ccc(F)cc2)c1/C=C/[C@@H](O)C[C@@H](O)c1nc(=O)o[nH]1]

This reaction was performed in the dark. Compound 26 (42 mg, 76 μmol) was dissolved in MeCN (0.5 mL) and 0.2M aqueous HCl (0.43 mL, 86 μmol) and the mixture was stirred at rt for 5.5 h. Next, the mixture was diluted with EtOAc (10 mL) and saturated aqueous NH₄Cl (10 mL). The layers were mixed, separated and the organic layer was washed with brine (2×5 mL), dried over Na₂SO₄ and filtered before concentration under reduced pressure. The residue was stripped with CH₂Cl₂ twice to yield 27 as a white foam (38 mg, 99%). ¹H NMR (400 MHz, CD₃OD) δ 7.73-7.67 (m, 2H), 7.20-7.14 (m, 2H), 6.67 (dd, J=16.1, 1.3 Hz, 1H), 5.56 (dd, J=16.1, 6.1 Hz, 1H), 4.52 (dd, J=7.6, 6.3 Hz, 1H), 4.36-4.29 (m, 1H), 3.54 (s, 3H), 3.52 (s, 3H), 3.51-3.43 (m, 1H), 1.97-1.78 (m, 2H), 1.29 (d, J=6.7 Hz, 6H).

### Example 6. Further Simvastatin Bioisosteres

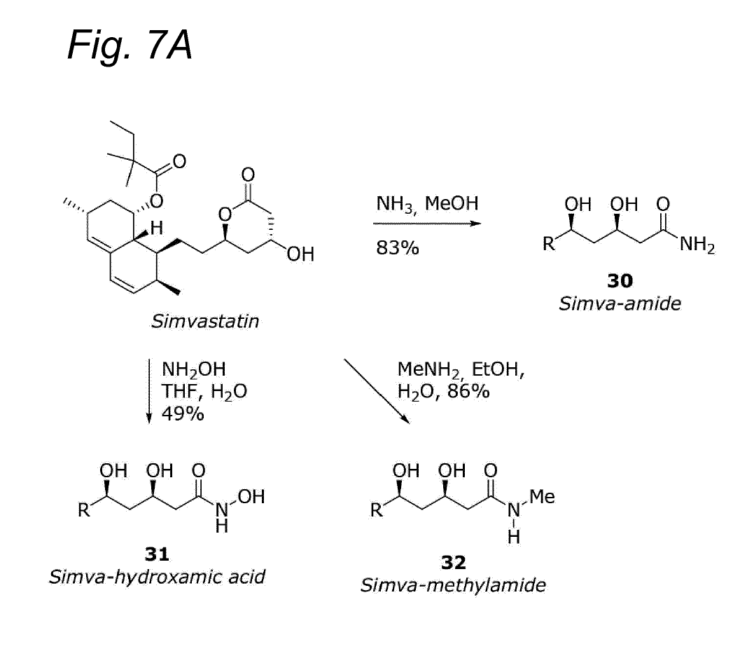

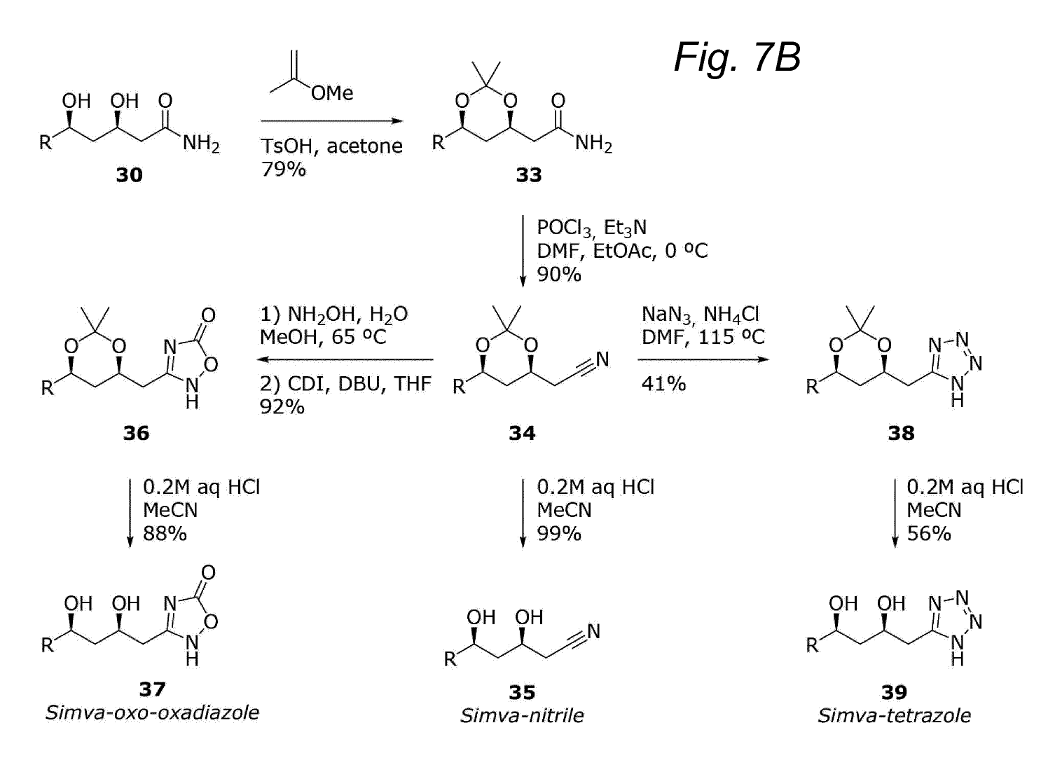

The syntheses of six simvastatin analogues were performed in an identical manner as described for the corresponding rosuvastatin analogues in Example 5 and FIGS. 3 and 4. The commercially available simvastatin was reacted with ammonia, hydroxylamine and methylamine to yield substrates 30, 31, and 32, respectively, in good to moderate yields (FIG. 7a).

After the diol moiety of amide 30 was protected, a dehydration was performed to prepare nitrile 34 (FIG. 7b). Acid mediated removal of the acetonide group provided substrate 35. Treatment of the nitrile with hydroxylamine followed by CDI provided oxo-oxadiazole 36 and reaction of the nitrile with sodium azide in the presence of ammonium chloride delivered the tetrazole motive (compound 38). Both heterocyclic compounds were deprotected, yielding Simvastatin analogues 37 and 39.

### (1S,3R,7S,8S,8aR)-8-((3R,5R)-7-amino-3,5-dihydroxy-7-oxoheptyl)-3,7-dimethyl-1,2,3,7,8,8a-hexahydronaphthalen-1-yl 2,2-dimethylbutanoate 30

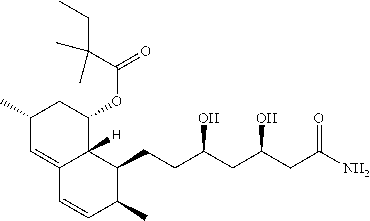 [SMILES: CCC(C)(C)C(=O)O[C@H]1C[C@@H](C)C=C2C=C[C@H](C)[C@H](CC[C@@H](O)C[C@@H](O)CC(N)=O)[C@H]21]

Simvastatin (25 mg, 60 μmol) was dissolved in ammonia (7N in MeOH, 427 μL, 2.99 mmol) and the solution was stirred at rt for 16 h. The mixture was concentrated under reduced pressure. Purification by flash chromatography (CH₂Cl₂:MeOH=95:5→90:10) afforded 30 as a colorless oil (22 mg, 83%). ¹H NMR (400 MHz, CDCl₃) δ 6.29 (br s, 1H), 5.98 (d, J=9.7 Hz, 1H), 5.78 (dd, J=9.6, 6.2 Hz, 1H), 5.55 (br s, 1H), 5.51-5.48 (m, 1H), 5.45-5.42 (m, 1H), 4.69 (br s, 1H), 4.18-4.27 (m, 1H), 3.84-3.76 (m, 1H), 3.62 (br s, 1H), 2.51-2.30 (m, 4H), 2.27-2.19 (m, 1H), 1.98 (ddd, J=15.0, 8.3, 2.5 Hz, 1H), 1.89 (dd, J=15.1, 3.5 Hz, 1H), 1.65-1.47 (m, 7H), 1.28-1.17 (m, 2H), 1.13 (s, 3H), 1.12 (s, 3H), 1.10 (d, J=7.4 Hz, 3H), 0.87 (d, J=7.0 Hz, 3H), 0.83 (t, J=7.5 Hz, 3H).

### (1S,3R,7S,8S,8aR)-8-((3R,5R)-3,5-dihydroxy-7-(hydroxyamino)-7-oxoheptyl)-3,7-dimethyl-1,2,3,7,8,8a-hexahydronaphthalen-1-yl 2,2-dimethylbutanoate 31

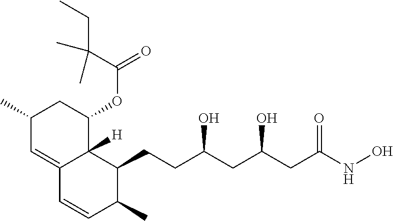 [SMILES: CCC(C)(C)C(=O)O[C@H]1C[C@@H](C)C=C2C=C[C@H](C)[C@H](CC[C@@H](O)C[C@@H](O)CC(=O)NO)[C@H]21]

Similar to example 1. To a solution of simvastatin (50 mg, 0.12 mmol) in THF (0.5 mL) was added hydroxylamine (50% in water, 20 μL, 0.60 mmol). The resulting mixture was stirred for 72 h. Next, the mixture was concentrated under reduced pressure. The residue was taken up in EtOAc (15 mL) and washed with saturated aqueous NH₄Cl (2×10 mL). After concentration under reduced pressure, the material was taken up in CH₂Cl₂ (10 mL), filtered over cotton wool, concentrated under reduced pressure and dried in vacuo to yield compound 31 (53 mg, 49%) as a colorless oil. ¹H NMR (400 MHz, CDCl₃) δ 10.00 (br s, 1H), 5.97 (d, J=9.7 Hz, 1H), 5.77 (dd, J=9.6, 6.1 Hz, 1H), 5.46-5.52 (m, 1H), 5.43-5.36 (m, 1H), 4.26 (br s, 1H), 3.76 (br s, 1H), 2.50-2.20 (m, 5H), 2.02-1.86 (m, 2H), 1.70-1.37 (m, 7H), 1.33-1.05 (m, 2H), 1.12 (s, 3H), 1.11 (s, 3H), 1.08 (d, J=7.4 Hz, 3H), 0.87 (d, J=6.9 Hz, 3H), 0.81 (d, J=7.5 Hz, 3H).

### (1S,3R,7S,8S,8aR)-8-((3R,5R)-3,5-dihydroxy-7-(methylamino)-7-oxoheptyl)-3,7-dimethyl-1,2,3,7,8,8a-hexahydronaphthalen-1-yl 2,2-dimethylbutanoate 32

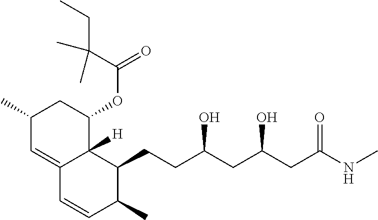

To a solution of simvastatin (25 mg, 60 μmol) in THF (0.5 mL) was added methanamine (33% in EtOH, 37 μL, 0.30 mmol) and the solution was stirred at rt for 16 h. Next, the mixture was concentrated under reduced pressure. Purification by flash chromatography (CH₂Cl₂:MeOH=95:5→90:10) afforded 32 as a colorless oil (23 mg, 86%). ¹H NMR (400 MHz, CDCl₃) δ 6.13 (br s, 1H), 5.98 (d, J=9.7 Hz, 1H), 5.78 (dd, J=9.6, 6.1 Hz, 1H), 5.49 (br s, 1H), 5.43 (q, J=3.0 Hz, 1H), 4.75 (br s, 1H), 4.26-4.15 (m, 1H), 3.79 (br s, 1H), 3.62 (br s, 1H), 2.82 (d, J=4.9 Hz, 2H), 2.50-2.19 (m, 5H), 1.98 (ABddd, J=15.1, 8.2, 2.6 Hz, 2H), 1.93-1.86 (m, 1H), 1.64-1.48 (m, 7H), 1.25-1.15 (m, 2H), 1.12 (s, 3H), 1.12 (s, 3H), 1.10 (d, J=7.4 Hz, 3H), 0.87 (d, J=7.0 Hz, 3H), 0.83 (t, J=7.5 Hz, 3H).

### (1S,3R,7S,8S,8aR)-8-(2-((4R,6R)-6-(2-amino-2-oxoethyl)-2,2-dimethyl-1,3-dioxan-4-yl)ethyl)-3,7-dimethyl-1,2,3,7,8,8a-hexahydronaphthalen-1-yl 2,2-dimethylbutanoate 33

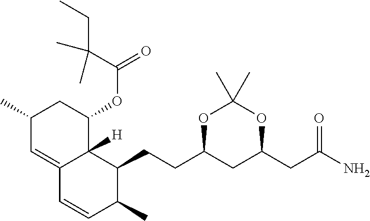 [SMILES: CCC(C)(C)C(=O)O[C@H]1C[C@@H](C)C=C2C=C[C@H](C)[C@H](CC[C@@H]3C[C@H](CC(N)=O)OC(C)(C)O3)[C@H]21]

A solution of 30 (520 mg, 1.19 mmol) in acetone (10 mL) was cooled to 4° C. Then, 2-methoxyprop-1-ene (343 μL, 3.58 mmol) and p-toluenesulfonic acid monohydrate (11.3 mg, 60 μmol) were added. The mixture was stirred at 4° C. for 30 min, then allowed to warm to room temperature and stirred for another 1 h. The reaction was quenched by the addition of saturated aqueous NaHCO₃ (20 mL) and the mixture was diluted with EtOAc (20 mL). The layers were mixed and separated, after which the aqueous phase was extracted with EtOAc (20 mL). The combined organic layers were washed with brine (20 mL), dried over Na₂SO₄ and filtered before concentration under reduced pressure. Purification by flash chromatography (CH₂Cl₂:MeOH=95:5) afforded 33 as a white foam (352 mg, 79%). ¹H NMR (400 MHz, CDCl₃) δ 6.31 (br s, 1H), 5.98 (d, J=9.6 Hz, 1H), 5.77 (dd, J=9.6, 6.1 Hz, 1H), 5.50 (br s, 1H), 5.37 (s, 1H), 5.34-5.31 (m, 1H), 4.25-4.17 (m, 1H), 3.80-3.72 (m, 1H), 2.48-2.32 (m, 4H), 2.27-2.20 (m, 1H), 2.02-1.88 (m, 2H), 1.70-1.32 (m, 7H), 1.45 (s, 3H), 1.39 (s, 3H), 1.29-1.15 (m, 2H), 1.12 (s, 3H), 1.11 (s, 3H), 1.07 (d, J=7.4 Hz, 3H), 0.87 (d, J=7.0 Hz, 3H), 0.83 (t, J=7.5 Hz, 3H).

### (1S,3R,7S,8S,8aR)-8-(2-((4R,6S)-6-(cyanomethyl)-2,2-dimethyl-1,3-dioxan-4-yl)ethyl)-3,7-dimethyl-1,2,3,7,8,8a-hexahydronaphthalen-1-yl 2,2-dimethyl butanoate 34

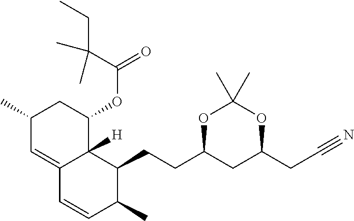 [SMILES: CCC(C)(C)C(=O)O[C@H]1C[C@@H](C)C=C2C=C[C@H](C)[C@H](CC[C@@H]3C[C@H](CC#N)OC(C)(C)O3)[C@H]21]

To a cooled (4° C.) solution of 33 (450 mg, 0.94 mmol) in dry EtOAc (9 mL) was added DMF (248 μL, 3.20 mmol) followed by Et₃N (419 μL, 3.01 mmol). Next, phosphoryl chloride (263 μL, 2.82 mmol) was added slowly. The resulting mixture was stirred at 4° C. for 30 min. The yellow mixture was diluted with saturated aqueous NaHCO₃ (15 mL) and EtOAC (15 mL) and warmed to rt. The layers were mixed, separated and the aqueous phase was extracted with EtOAc (10 mL). The combined organic layers were washed with saturated aqueous NaHCO₃ (15 mL) and brine (2×15 mL), dried over Na₂SO₄ and filtered before concentration under reduced pressure. Purification by flash chromatography (heptane:EtOAc=3:1) afforded 34 as a yellow oil (387 mg, 90%). ¹H NMR (400 MHz, CDCl₃) δ 5.98 (d, J=9.6 Hz, 1H), 5.78 (dd, J=9.7, 6.1 Hz, 1H), 5.50 (br s, 1H), 5.36-5.31 (m, 1H), 4.13-4.05 (m, 1H), 3.78-3.69 (m, 1H), 2.55-2.32 (m, 4H), 2.27-2.20 (m, 1H), 2.02-1.89 (m, 2H), 1.72-1.47 (m, 7H), 1.43 (s, 3H), 1.38 (s, 3H), 1.30-1.14 (m, 2H), 1.13 (s, 3H), 1.12 (s, 3H), 1.08 (d, J=7.4 Hz, 3H), 0.87 (d, J=7.0 Hz, 3H), 0.84 (t, J=7.5 Hz, 3H).

### (1S,3R,7S,8S,8aR)-8-((3R,5S)-6-cyano-3,5-dihydroxyhexyl)-3,7-dimethyl-1,2,3,7,8,8a-hexahydronaphthalen-1-yl 2,2-dimethylbutanoate 35

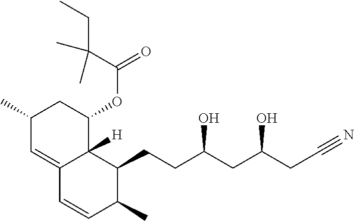 [SMILES: CCC(C)(C)C(=O)O[C@H]1C[C@@H](C)C=C2C=C[C@H](C)[C@H](CC[C@@H](O)C[C@@H](O)CC#N)[C@H]21]

To a solution of 34 (52 mg, 0.11 mmol) in MeCN (0.5 mL) was added 0.2 M aqueous HCl (553 μL, 0.11 mmol). The mixture was stirred at rt for 4 h. Then, the mixture was diluted with EtOAc (10 mL) and saturated aqueous NaHCO₃ (5 mL). The layers were mixed, separated and the aqueous phase was extracted with EtOAc (2×5 mL). The combined organic layers were washed with brine (5 mL), dried over Na₂SO₄ and filtered before concentration under reduced pressure. The residue was stripped with CH₂Cl₂ (2×2 mL) to yield compound 35 as a pale yellow oil (45 mg, 99%).

### (1S,3R,7S,8S,8aR)-8-((3R,5)-3,5-dihydroxy-6-(5-oxo-2,5-dihydro-1,2,4-oxadiazol-3-yl)hexyl)-3,7-dimethyl-1,2,3,7,8,8a-hexahydronaphthalen-1-yl 2,2-dimethylbutanoate 37

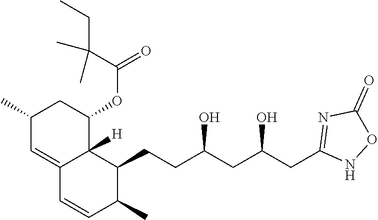 [SMILES: CCC(C)(C)C(=O)O[C@H]1C[C@@H](C)C=C2C=C[C@H](C)[C@H](CC[C@@H](O)C[C@@H](O)Cc3nc(=O)o[nH]3)[C@H]21]

To a solution of 34 (97 mg, 0.21 mmol) in MeOH (1 mL) was added hydroxylamine (50% in water, 204 μL, 3.09 mmol). The resulting mixture was warmed to 65° C. and stirred for 5 h. After allowing it to cool to rt, the mixture was concentrated under reduced pressure. The residue was stripped twice with 2 mL of THF. The residue was dissolved in THF (1 mL) and CDI (50 mg, 0.31 mmol) was added. The resulting mixture was stirred for 10 min, before adding DBU (39 μL, 0.26 mmol). The mixture was stirred for 16 h and more CDI (100 mg) and DBU (40 μL) were added. After 2 h the mixture was diluted with saturated aqueous NH₄Cl (15 mL) and EtOAc (15 mL). The layers were mixed, separated and the organic phase was washed with brine (10 mL), dried over Na₂SO₄ and filtered before concentration under reduced pressure. Purification by flash chromatography (heptane:EtOAc=2:1→1:3) afforded 36 as a yellow oil (98 mg).

Compound 36 (98 mg, 0.19 mmol) was dissolved in MeCN (950 μL) and 0.2M aqueous HCl (948 μL, 0.19 mmol) and the mixture was stirred at rt for 3.5 h. Then, the mixture was diluted with EtOAc (10 mL) and saturated aqueous NH₄Cl (10 mL). The layers were mixed, separated and the organic layer was washed with brine (5 mL), dried over Na₂SO₄ and filtered before concentration under reduced pressure. Purification by flash chromatography (CH₂Cl₂:MeOH=100:2.5→100:10) afforded 37 as a white foam (80 mg, 88%). ¹H NMR (400 MHz, CDCl₃) δ 5.99 (d, J=9.7 Hz, 1H), 5.76 (dd, J=9.6, 6.2 Hz, 1H), 5.55-5.49 (m, 2H), 4.23-4.15 (m, 1H), 3.86-3.78 (m, 1H), 3.38 (br s, 1H), 2.78 (dd, J=15.2, 3.4 Hz, 1H), 2.62 (dd, J=15.2, 7.4 Hz, 1H), 2.53-2.43 (m, 1H), 2.20-2.35 (m, 2H), 2.02 (ddd, J=15.0, 8.7, 2.5 Hz, 1H), 1.88-1.77 (m, 2H), 1.63-1.46 (m, 6H), 1.37-1.17 (m, 2H), 1.15-1.09 (m, 9H), 0.86 (d, J=7.0 Hz, 3H), 0.83 (t, J=7.5 Hz, 3H).

### (1S,3R,7S,8S,8aR)-8-((3R,5S)-3,5-dihydroxy-6-(1H-tetrazol-5-yl)hexyl)-3,7-dimethyl-1,2,3,7,8,8a-hexahydronaphthalen-1-yl 2,2-dimethylbutanoate 39

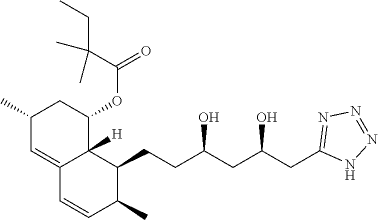 [SMILES: CCC(C)(C)C(=O)O[C@H]1C[C@@H](C)C=C2C=C[C@H](C)[C@H](CC[C@@H](O)C[C@@H](O)Cc3nnn[nH]3)[C@H]21]

To a solution of 34 (73 mg, 0.15 mmol) in DMF (0.6 mL) were added NH₄Cl (83 mg, 1.55 mmol) and sodium azide (101 mg, 1.55 mmol). The resulting mixture was warmed to 115° C. and stirred for 22 h. The mixture was diluted with EtOAc (15 mL) and saturated aqueous NH₄Cl (10 mL). The layers were mixed and separated and the aqueous phase was extracted with EtOAc (10 mL) and the combined organic layers were washed with brine (10 mL), dried over Na₂SO₄ and filtered before concentration under reduced pressure. Purification by flash chromatography (CH₂Cl₂:MeOH=100:2.5→100:10) afforded 38 (32 mg).

Compound 38 (32 mg, 64 μmol) was dissolved in MeCN (320 μL) and 0.2M aqueous HCl (320 μL, 64 μmol) and the mixture was stirred at rt for 3.5 h. Then, the mixture was diluted with EtOAc (10 mL) and saturated aq. NH₄Cl (10 mL). The layers were mixed, separated and the aqueous phase was extracted with EtOAc (5 mL). The combined organic layers were dried over Na₂SO₄ and filtered before concentration under reduced pressure. Purification by flash chromatography (CH₂Cl₂:MeOH=100:5→100:10) afforded 39 as a colorless oil (18 mg, 56%). ¹H NMR (400 MHz, CDCl₃) δ 5.99 (d, J=9.7 Hz, 1H), 5.76 (dd, J=9.6, 6.0 Hz, 1H), 5.55-5.49 (m, 2H), 4.27-4.19 (m, 1H), 3.87-3.79 (m, 1H), 3.23 (dd, J=15.4, 3.8 Hz, 1H), 3.07 (dd, J=15.5, 7.6 Hz, 1H), 2.53-2.43 (m, 1H), 2.37-2.20 (m, 2H), 2.02 (ddd, J=15.0, 8.7, 2.5 Hz, 1H), 1.87-1.73 (m, 2H), 1.69-1.45 (m, 6H), 1.36-1.01 (m, 11H), 0.91-0.78 (m, 6H).

### Example 7. Lovastatin Analogues

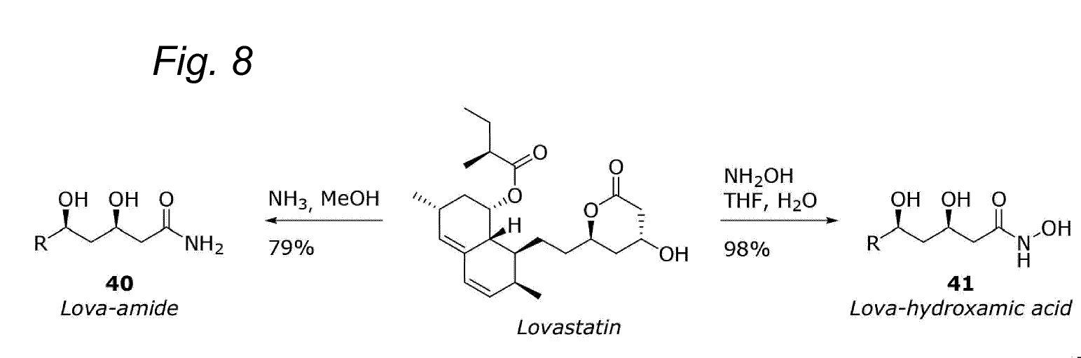

The amide and hydroxamic acid analogues of lovastatin were prepared using the exact same strategy as applied for the simvastatin derivatives (FIG. 8). The commercially available lovastatin was reacted with ammonia to obtain the corresponding amide 40, whereas reaction with hydroxylamine provided hydroxamic acid 41, both in a high yield.

### (1S,3R,7S,8S,8aR)-8-((3R,5R)-7-amino-3,5-dihydroxy-7-oxoheptyl)-3,7-dimethyl-1,2,3,7,8,8a-hexahydronaphthalen-1-yl (S)-2-methylbutanoate 40

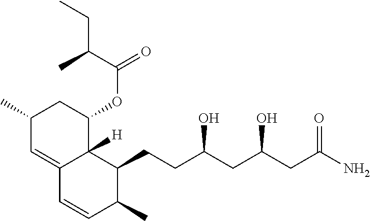 [SMILES: CC[C@H](C)C(=O)O[C@H]1C[C@@H](C)C=C2C=C[C@H](C)[C@H](CC[C@@H](O)C[C@@H](O)CC(N)=O)[C@H]21]

Lovastatin (50 mg, 124 gmol) was dissolved in ammonia (7N in MeOH, 883 μL, 6.18 mmol) and the solution was stirred at rt for 24 h. The mixture was concentrated under reduced pressure. Purification by flash chromatography (CH₂Cl₂:MeOH=95:5→90:10) afforded 40 as a colorless oil (41 mg, 79%). ¹H NMR (400 MHz, CDCl₃) δ 6.24 (br s, 1H), 5.99 (d, J=9.7 Hz, 1H), 5.79 (dd, J=9.6, 6.1 Hz, 1H), 5.56 (br s, 1H), 5.53-5.50 (m, 1H), 5.45-5.41 (m, 1H), 4.63 (br s, 1H), 4.27-4.18 (m, 1H), 3.86-3.76 (m, 1H), 3.49 (br s, 1H), 2.51-2.20 (m, 6H), 1.98-1.89 (m, 2H), 1.72-1.51 (m, 6H), 1.50-1.36 (m, 1H), 1.28-1.15 (m, 2H), 1.11 (d, J=6.9 Hz, 3H), 1.09 (d, J=7.4 Hz, 3H), 0.91-0.86 (m, 6H).

### (1S,3R,7S,8S,8aR)-8-((3R,5R)-3,5-dihydroxy-7-(hydroxyamino)-7-oxoheptyl)-3,7-dimethyl-1,2,3,7,8,8a-hexahydronaphthalen-1-yl (S)-2-methylbutanoate 41

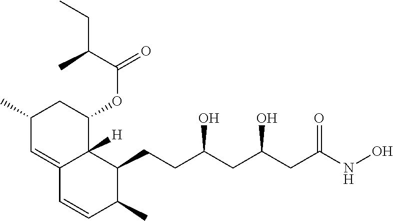 [SMILES: CC[C@H](C)C(=O)O[C@H]1C[C@@H](C)C=C2C=C[C@H](C)[C@H](CC[C@@H](O)C[C@@H](O)CC(=O)NO)[C@H]21]

To a solution of lovastatin (50 mg, 0.12 mmol) in THF (0.5 mL) was added hydroxylamine (50% in water, 20 μL, 0.62 mmol). The resulting mixture was stirred for 72 h. Next, the mixture was concentrated under reduced pressure, stripped with CHCl₃ and dried in vacuo to yield compound 41 (53 mg, 98%) as a white foam. ¹H NMR (400 MHz, CDCl₃) δ 5.98 (d, J=9.7 Hz, 1H), 5.78 (dd, J=9.4, 6.1 Hz, 1H), 5.54-5.48 (m, 1H), 5.46-5.39 (m, 1H), 4.29-4.20 (m, 1H), 3.83-3.72 (m, 1H), 2.53-2.19 (m, 6H), 1.99-1.89 (m, 2H), 1.72-1.37 (m, 7H), 1.33-1.12 (m, 2H), 1.10 (d, J=6.9 Hz, 3H), 1.08 (d, J=7.4 Hz, 3H), 0.93-0.82 (m, 6H).

### Example 8. Atorvastatin Analogues

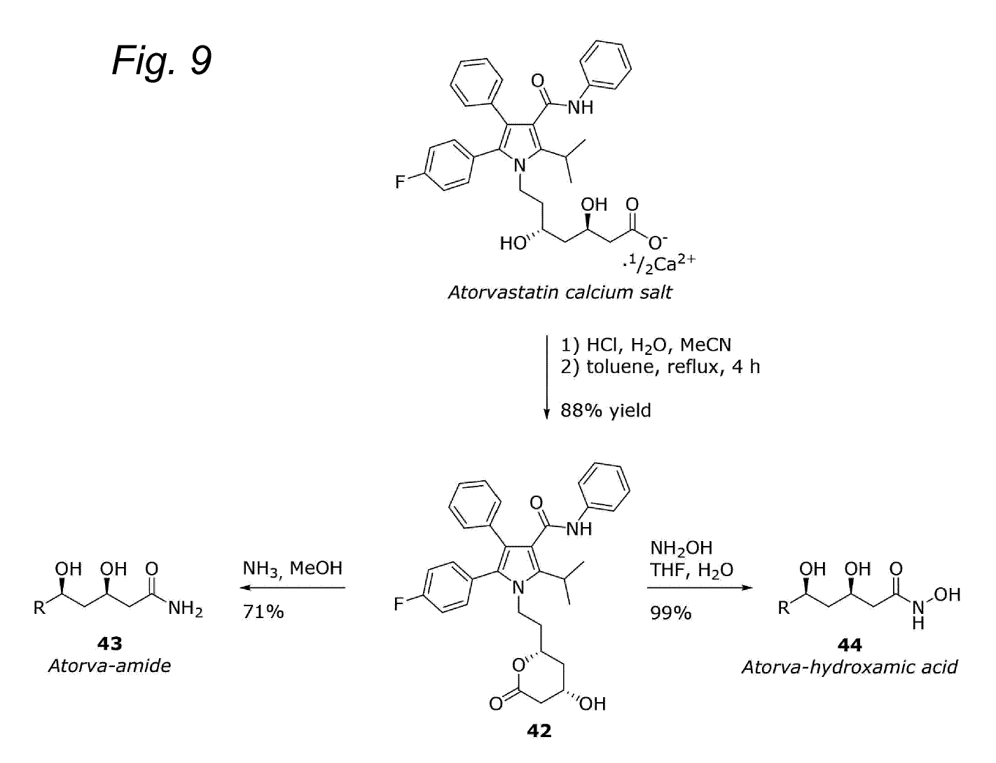

The commercially available calcium salt of atorvastatin was converted into the lactone form in the way as described for Rosuvastatin (see FIG. 3). With lactone 42 in hand, an identical strategy as before was applied to obtain amide 43 and hydroxamic acid 44 (FIG. 9).

### 5-(4-fluorophenyl)-1-(2-((2R,4S)-4-hydroxy-6-oxotetrahydro-2H-pyran-2-yl)ethyl)-2-isopropyl-N,4-diphenyl-1H-pyrrole-3-carboxamide 42

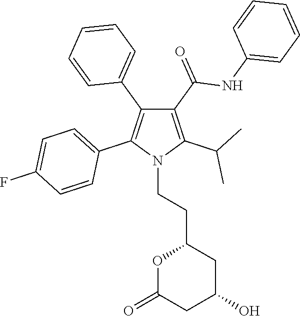 [SMILES: CC(C)c1c(C(=O)Nc2ccccc2)c(-c2ccccc2)c(-c2ccc(F)cc2)n1CC[C@@H]1C[C@H](O)CC(=O)O1]

Atorvastatin calcium salt (500 mg, 0.413 mmol) was suspended in water (8 mL) and EtOAc (4 mL) and cooled in an ice-bath (4° C.). Next, 0.2M aqueous HCl (4.55 mL, 0.910 mmol) was added dropwise under vigorous stirring, which resulted in a clear solution. The mixture was allowed to warm to rt, the layers were separated and the aqueous phase was extracted with EtOAc (3×5 mL). The combined organic layers were washed with brine (5 mL), dried over Na₂SO₄ and filtered before concentration under reduced pressure. The resulting foam (469 mg) was dissolved in toluene (20 mL) and the mixture was heated to reflux under Dean-Stark conditions for 2.5 h. The mixture was allowed to cool to rt and concentrated under reduced pressure. Purification by flash chromatography (heptane:EtOAc=1:3→1:5) afforded 42 as a white foam (394 mg, 88%). ¹H NMR (400 MHz, CDCl₃) δ 7.24-7.10 (m, 9H), 7.09-6.96 (m, 5H), 6.92-6.82 (br s, 1H), 4.57-4.47 (m, 1H), 4.34-4.27 (m, 1H), 4.27-4.16 (m, 1H), 4.09-3.97 (m, 1H), 3.62-3.47 (m, 1H), 2.66 (ABdd, J=17.7, 4.8 Hz, 1H), 2.55 (ABddd, J=17.7, 3.4, 1.5 Hz, 1H), 2.11 (d, J=2.9 Hz, 1H), 1.95-1.82 (m, 1H), 1.81-1.67 (m, 2H), 1.63-1.57 (m, 1H), 1.56-1.45 (m, 6H).

### 1-((3R,5R)-7-amino-3,5-dihydroxy-7-oxoheptyl)-5-(4-fluorophenyl)-2-isopropyl-N,4-diphenyl-1H-pyrrole-3-carboxamide 43

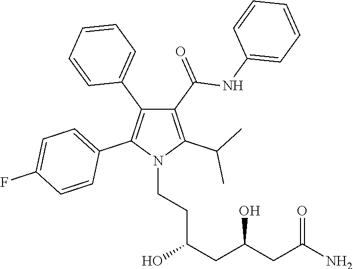 [SMILES: CC(C)c1c(C(=O)Nc2ccccc2)c(-c2ccccc2)c(-c2ccc(F)cc2)n1CC[C@@H](O)C[C@@H](O)CC(N)=O]

Compound 42 (100 mg, 185 μmol) was dissolved in ammonia (7N in MeOH, 1.32 mL, 6.18 mmol) and the solution was stirred at rt for 24 h. The mixture was concentrated under reduced pressure. Purification by flash chromatography (CH₂Cl₂:MeOH=95:5→90:10) afforded 43 as a white foam (73 mg, 71%). ¹H NMR (400 MHz, CDCl₃) δ 7.24-6.95 (m, 14H), 6.88 (br s, 1H), 5.73 (br s, 1H), 5.44 (br s, 1H), 4.42 (br s, 1H), 4.20-4.05 (m, 2H), 4.01-3.90 (m, 1H), 3.79-3.71 (m, 1H), 3.63-3.48 (m, 2H), 2.34-2.23 (m, 2H), 1.74-1.41 (m, 9H), 1.28-1.14 (m, 1H).

### 1-((3R,5R)-3,5-dihydroxy-7-(hydroxyamino)-7-oxoheptyl)-5-(4-fluorophenyl)-2-isopropyl-N,4-diphenyl-1H-pyrrole-3-carboxamide 44

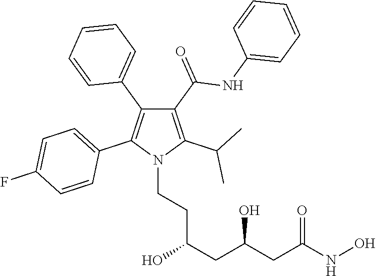 [SMILES: CC(C)c1c(C(=O)Nc2ccccc2)c(-c2ccccc2)c(-c2ccc(F)cc2)n1CC[C@@H](O)C[C@@H](O)CC(=O)NO]

To a solution of 42 (51 mg, 94 gmol) in THF (0.5 mL) was added hydroxylamine (50% in water, 16 μL, 0.47 mmol). The resulting mixture was stirred for 48 h. Next, the mixture was concentrated under reduced pressure, stripped with CH₂Cl₂ (2×10 mL) and dried in vacuo to yield 44 (53 mg, 99%) as a white foam. ¹H NMR (400 MHz, CDCl₃) δ 7.22-6.79 (m, 14H), 4.22-3.82 (m, 3H), 3.70-3.40 (m, 2H), 2.28-2.02 (m, 2H), 1.70-1.08 (m, 10H).

### Example 9. Fluvastatin Analogues

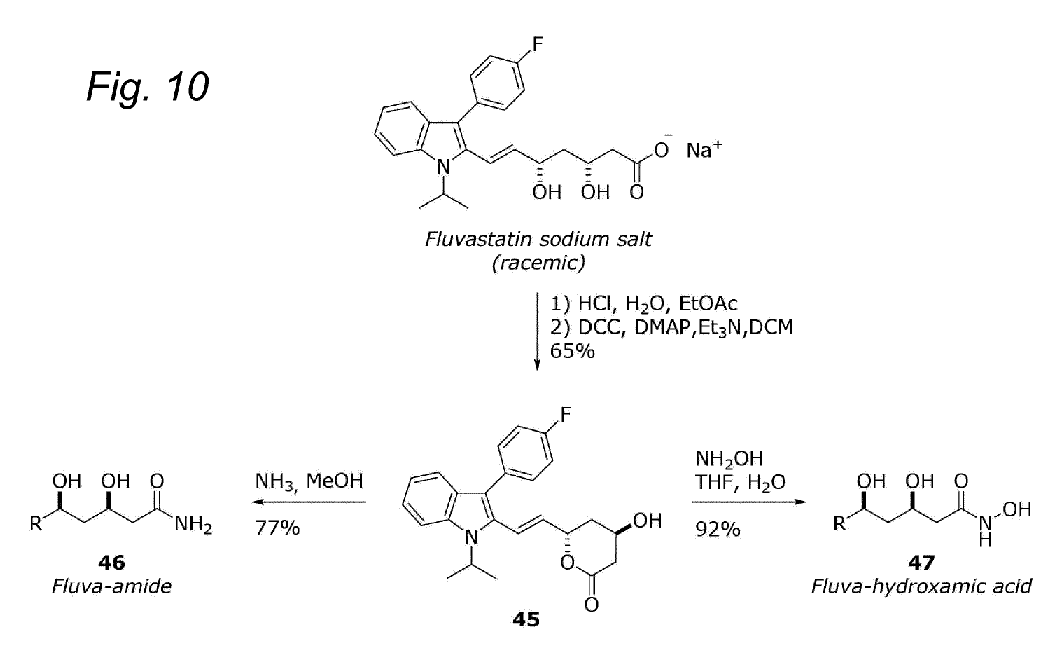

The sodium salt of racemic fluvastatin was transformed into its lactone form by activation of the carboxylic acid with N,N-dicyclohexylcarbodiimide (DCC) (FIG. 10). With lactone 45 in hand, again the same strategy as before was applied to obtain amide 46 and hydroxamic acid 47.

### (4R,6S)-6-((E)-2-(3-(4-fluorophenyl)-1-isopropyl-1H-indol-2-yl)vinyl)-4-hydroxytetrahydro-2H-pyran-2-one 45

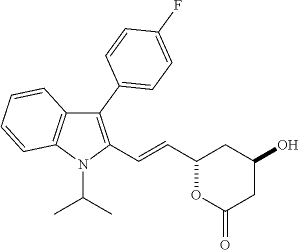 [SMILES: CC(C)n1c(/C=C/[C@@H]2C[C@@H](O)CC(=O)O2)c(-c2ccc(F)cc2)c2ccccc21]

This reaction was performed in the dark. Fluvastatin sodium salt (250 mg, 0.58 mmol) was suspended in water (6 mL) and EtOAc (6 mL) and cooled in an ice-bath. 0.2M aqueous HCl (3.00 mL, 0.60 mmol) was added dropwise under vigorous stirring. Next, the mixture was allowed to warm to rt. The layers were separated and the aqueous phase was extracted with EtOAc (3×6 mL). The combined organic layers were washed with brine (6 mL), dried over Na₂SO₄ and filtered before concentration under reduced pressure. The residue was dissolved in CH₂Cl₂ (6 mL) and cooled in an ice-bath. DCC (125 mg, 0.61 mmol), Et₃N (88 μL, 0.63 mmol) and DMAP (3.5 mg, 29 μmol) were added. The mixture was allowed to warm to rt and stirred for 16 h. The formed precipitate was removed by filtration. The filtrate was diluted with EtOAc (20 mL) and washed with 10% aqueous citric acid (2×15 mL), saturated aqueous NaHCO₃ (15 mL) and brine (10 mL), dried over Na₂SO₄ and filtered before concentration under reduced pressure. Purification by flash chromatography (heptane:EtOAc=1:1→1:5) afforded 45 as a white foam (149 mg, 65%). ¹H NMR (400 MHz, CDCl₃) δ 7.57-7.49 (m, 2H), 7.42-7.35 (m, 2H), 7.24-7.17 (m, 1H), 7.15-7.04 (m, 3H), 6.76 (dd, J=16.0, 1.4 Hz, 1H), 5.68 (dd, J=16.0, 6.0 Hz, 1H), 5.29-5.21 (m, 1H), 4.83 (hept, J=7.2 Hz, 1H), 4.39-4.32 (m, 1H), 2.75 (ABdd, J=17.8, 4.8 Hz, 1H), 2.64 (ABddd, J=17.8, 3.7, 1.5 Hz, 1H), 2.02 (d, J=3.1 Hz, 1H), 1.97-1.89 (m, 1H), 1.75-1.63 (m, 7H).

### (3R,5S,E)-7-(3-(4-fluorophenyl)-1-isopropyl-1H-indol-2-yl)-3,5-dihydroxyhept-6-enamide 46

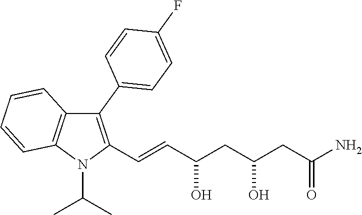 [SMILES: CC(C)n1c(/C=C/[C@@H](O)C[C@@H](O)CC(N)=O)c(-c2ccc(F)cc2)c2ccccc21]

This reaction was performed in the dark. Lactone 45 (91 mg, 0.23 mmol) was dissolved in ammonia (7N in MeOH, 1.65 mL, 11.5 mmol) and the solution was stirred at rt for 24 h. Next, the mixture was concentrated under reduced pressure. Purification by flash chromatography (CH₂Cl₂:MeOH=95:5→85:15) afforded 46 as a white foam (73 mg, 77%). ¹H NMR (400 MHz, CDCl₃) δ 7.56-7.48 (m, 2H), 7.43-7.35 (m, 2H), 7.22-7.15 (m, 1H), 7.13-7.04 (m, 3H), 6.69 (dd, J=16.0, 1.4 Hz, 1H), 5.79 (br s, 1H), 5.69 (dd, J=16.0, 5.5 Hz, 1H), 5.48 (br s, 1H), 4.91-4.78 (m, 1H), 4.55-4.46 (m, 1H), 4.43 (br s, 1H), 4.27-4.18 (m, 1H), 3.46 (br s, 1H), 2.44-2.30 (m, 2H), 1.73-1.57 (m, 7H), 1.48 (m, 1H).

### (3R,5S,E)-7-(3-(4-fluorophenyl)-1-isopropyl-1H-indol-2-yl)-N,3,5-trihydroxyhept-6-enamide 47

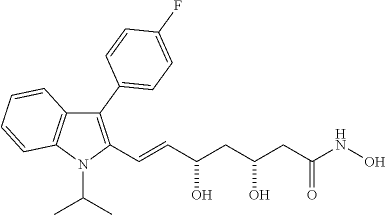 [SMILES: CC(C)n1c(/C=C/[C@@H](O)C[C@@H](O)CC(=O)NO)c(-c2ccc(F)cc2)c2ccccc21]

To a solution of 45 (50 mg, 0.12 mmol) in THF (0.5 mL) was added hydroxylamine (50% in water, 21 μL, 0.63 mmol). The resulting mixture was stirred for 24 h. Next, the mixture was concentrated under reduced pressure, stripped with CH₂Cl₂ (2×5 mL) and dried in vacuo to yield 47 (50 mg, 92%) as a white foam. ¹H NMR (400 MHz, CDCl₃) δ 7.54-7.46 (m, 2H), 7.41-7.32 (m, 2H), 7.19-7.13 (m, 1H), 7.09-7.01 (m, 3H), 6.62 (d, J=15.8 Hz, 1H), 5.65 (dd, J=16.0, 5.7 Hz, 1H), 4.84-4.72 (m, 1H), 4.40 (br s, 1H), 4.20 (br s, 1H), 2.38-2.26 (m, 2H), 1.80-1.05 (m, 8H).

### Example 10. Characteristics of New Statin Bioisosteres

The Complex III inhibitory activity of various newly synthesized statin derivatives described above was tested at 100 μM, as per the assay described in Example 2. Results of this assay are shown in Table 4. Statistical analysis: Complex III enzyme activity values were compared to vehicle control levels using one-way ANOVA with Dunnett's post-hoc correction analysis. Mean±SEM; n=3 independent experiments.

The HMG-CoA reductase (HMGR) activity of various newly synthesized statin derivatives described above was also determined, at 300 nM as per the assay described in Example 3. These results are shown in Table 4. Statistical analysis: HMG-CoA reductase enzyme activity values were compared to vehicle control levels using one-way ANOVA with Dunnett's post-hoc correction analysis. Mean±SEM; n=3 independent experiments.

TABLE 4
effect of statin analogues

| | | HMGR activity | complex III activity | | |
|---|---|---|---|---|---|
| | | (% of control) | (% of control) | Significance | Significance |
| | | Mean (SEM) | Mean (SEM) | HMGR | complex III |
| control | | 100.00 (5.31) | 100.00 (8.27) | | |
| simvastatin | carboxylic acid | 63.10 (10.70) | — | * | |
| | lactone | — | 44.55 (6.27) | | *** |
| | amide | 110.10 (11.81) | 73.77 (7.57) | ### | # |
| | methylamide | 92.40 (8.84) | 50.43 (5.71) | | *** |
| | nitrile | 85.05 (10.36) | 53.24 (1.53) | | *** |
| | hydroxamic acid | 38.83 (5.19) | 68.59 (5.73) | *** | ** |
| | oxo-oxadiazole | 73.44 (2.81) | 74.37 (4.20) | | *# |
| | tetrazole | 72.43 (4.97) | 80.37 (5.13) | | ## |
| rosuvastatin | carboxylic acid | 24.09 (3.30) | — | *** | |
| | lactone | — | 86.28 (2.62) | | |
| | amide | 81.57 (10.29) | 94.39 (6.41) | ### | |
| | methylamide | 82.82 (12.67) | 83.54 (5.98) | ### | |
| | nitrile | 102.50 (7.87) | 95.96 (11.43) | ### | |
| | dimethylamide | 91.35 (15.32) | 88.90 (7.89) | ### | |
| | hydroxamic acid | 72.81 (9.03) | 90.25 (7.98) | *### | |
| | oxo-oxadiazole | 88.17 (4.21) | 85.85 (10.62) | ### | |
| | tetrazole | 81.28 (4.70) | 85.24 (4.09) | ### | |
| | nor-tetrazole | 86.12 (5.73) | 134.50 (17.29) | ### | **## |
| | nor-oxo-oxadiazole | 100.30 (7.82)^{a} | 143.50 (15.67) | ### | ## |
| atorvastatin | carboxylic acid | 24.39 (8.91) | — | *** | |
| | lactone | — | 80.98 (3.75) | | |
| | amide | 97.37 (3.89) | 54.34 (7.70) | ### | |
| | hydroxamic acid | 60.87 (4.50) | 105.60 (13.57) | **## | |
| lovastatin | carboxylic acid | 68.18 (14.81) | — | * | |
| | lactone | — | 63.21 (3.31) | | ** |
| | amide | 102.30 (10.00) | 104.10 (12.47) | | # |
| | hydroxamic acid | 70.19 (6.79) | 92.95 (10.32) | * | |

^{a}raw data showed an unexpected spread ranging from 49 to 124 for this compound - repeat of this experiment at a larger scale to improve precision gave a result of 65.69 +/− 2.29% (n = 1); in light of this, more repeats of all entries in this table are planned;
significance as compared by one-way ANOVA with Dunnett's post-hoc analysis (compared to control): *p < 0.05, **p < 0.01, ***p < 0.001;
significance (compared to reference; lactone for complex III, carboxylic acid for HMGR): # p < 0.05, ## p < 0.01, ### p < 0.001;
Mean ± SEM;
n = 3 independent experiments

### REFERENCE LIST

1. Tom J. J. Schirris, G. Herma Renkema, Tina Ritschel et al., Cell Metabolism (2015) 22:399-407.
2. Marcoff, L., and Thompson, P. D. (2007). J. Am. Coll. Cardiol. 49, 2231-2237.
3. Sirvent, P., Mercier, J., and Lacampagne, A. (2008). Curr. Opin. Pharmacol. 8, 333-338.
4. A. Endo, M. Kuroda, K. Tanzawa, FEBS Lett. 72, 323 (1976).
5. K. M. Bischoff, V. W. Rodwell, Biochem. Med. Metab. Biol. 48, 149 (1992).
6. Wood, D. J., Vlieg, J. D., Wagener, M., & Ritschel, T. (2012). Journal of chemical information and modeling, 52(8), 2031-2043.
7. Traci Turner and Evan A. Stein, Clinical Therapeutics (2015). DOI: 10.1016/j.clinthera.2015.09.004
8. Sahebkar and Watts, Clinical Therapeutics (2013) 35(8): pages 1082-1098. doi: 10.1016/j.clinthera.2013.06.019
9. Dröse S, and Brandt U., Adv. Exp. Med. Biol. (2012) 748: pages 145-169 doi: 10.1007/978-1-4614-3573-0_6
10. Prueksaritanont, T., Subramanian, R., Fang, X., Ma, B., et al. (2002) Drug Metab. Dispos. 30, 505-512.
11. K. Kajinami, N. Takekoshi, Y. Saito (2003) Cardiovascular Drug Reviews Vol. 21, No. 3, pp. 199-215.
12. E S Istvan and J Deisenhofer, Science (2001) 292(5519):1160-1164.
13. Haines, B. E., Wiest O, and Stauffacher C. V. (2013) Accounts of chemical research 46:11 2416-2426.

What is claimed is:

1. A compound that inhibits 3-hydroxy-3-methylglutaryl-coenzyme A (HMG-CoA) reductase while not inhibiting mitochondrial complex III, wherein said compound has the general formula I: 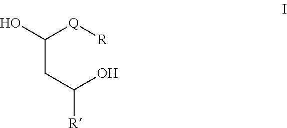
wherein Q is selected from —CH₂— or a bond,
wherein R′ is selected from the group consisting of: 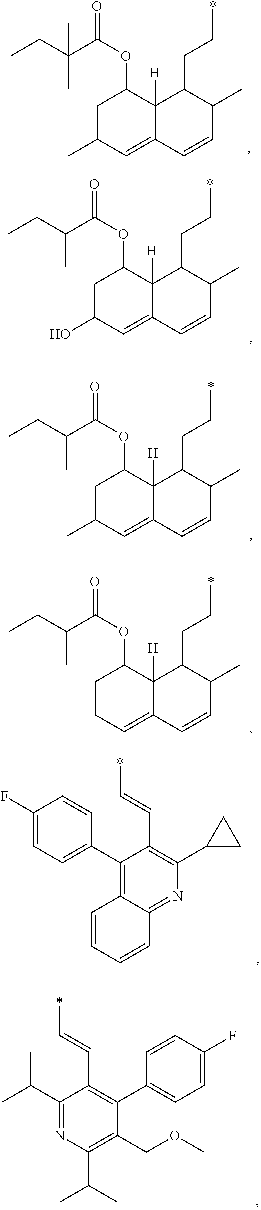 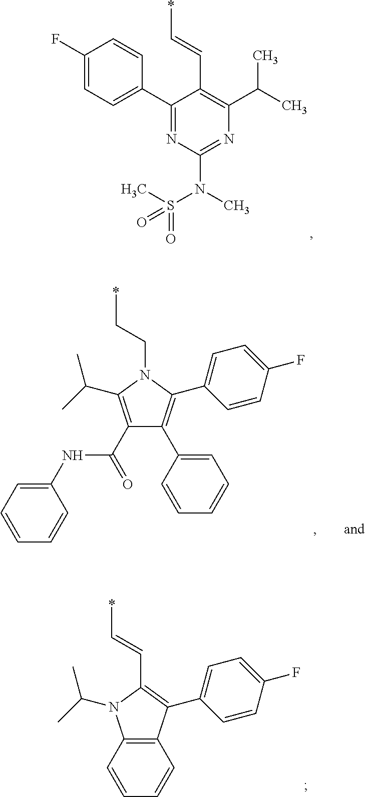
wherein R is a substituted or non-substituted moiety selected from the group consisting of hydroxamic acid, tetrazole, nitro, thiocarboxylic acid, amide, sulfonic acid, sulfonamide, phosphonate, boronic acid, 4-linked 3-hydroxycyclobut-3-ene-1,2-dione, an oxo-oxadiazole-like moiety with optional sulfur-substitutions, and moieties having a general formula II or III: 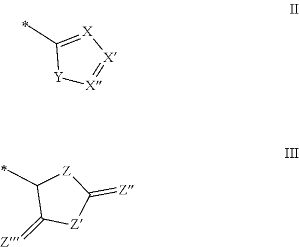
wherein X and X′ and X″ are each individually N, CH, or C—OH,
wherein Y is O, S, NH, or N—CH₃,
wherein Z, Z′, Z″, and Z′″ are each individually O, S, NH, or CH₂.

2. The compound according to claim 1, wherein R is selected from the group consisting of: 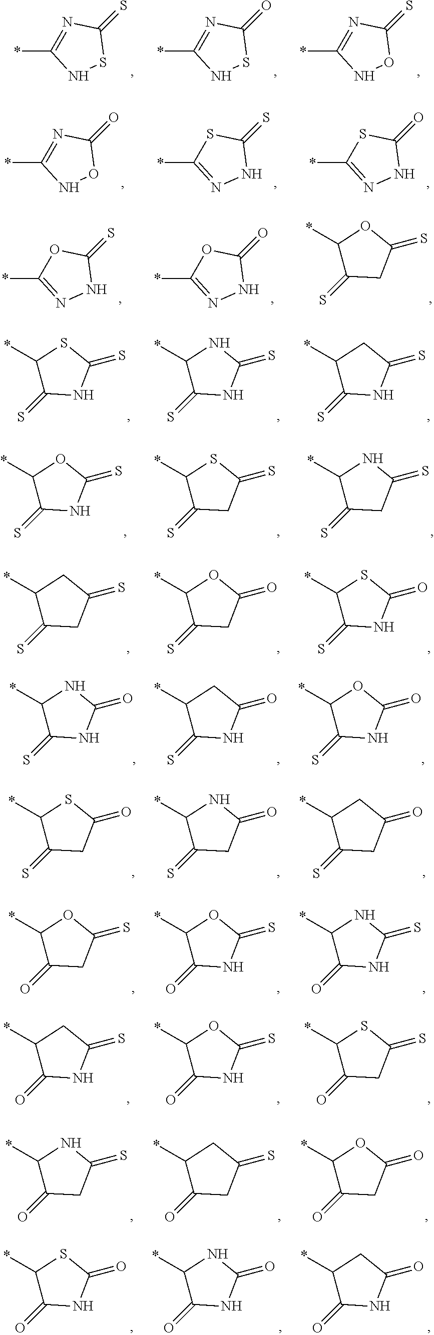 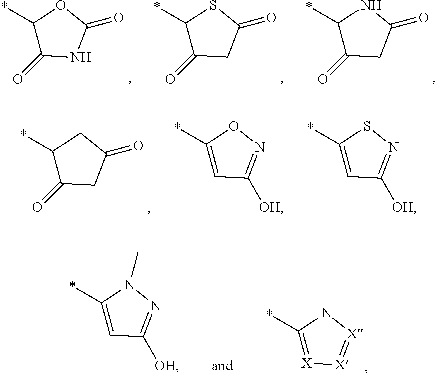
wherein X, X′, and X″ are as defined in claim 1.

3. The compound according to claim 1, wherein the compound is derived from a compound selected from the group consisting of simvastatin, atorvastatin, cerivastatin, fluvastatin, lovastatin, mevastatin, pitavastatin, pravastatin, and rosuvastatin, wherein the carboxylic acid moiety has been replaced by a substituted or non-substituted moiety R as defined in claim 1.

4. A pharmaceutical composition comprising a compound according to claim 1, further comprising a pharmaceutically acceptable excipient.

5. The pharmaceutical composition according to claim 4, in the form of a tablet, soft or hard capsule, ampoule, solution for injection, emulsion for injection, suspension for injection, solution for inhalation, emulsion for inhalation, suspension for inhalation, cream, or ointment.

6-14. (canceled)

15. The compound according to claim 1, wherein Z″ and Z′″ are each individually O or S.

16. The compound according to claim 2, wherein Q is a bond.

17. The compound according to claim 1, wherein the compound is of general formula Ia or Ie, 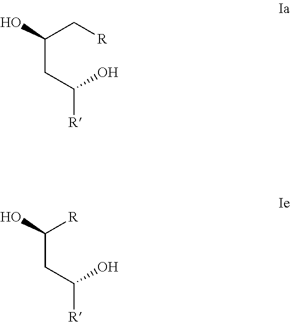
wherein R is an oxo-oxadiazole-like moiety or of general formula II or III as defined in claim 1, wherein X, X′, and X″ are as defined in claim 1.

18. The compound according to claim 17, wherein the compound has the general formula Ie.

19. A method for the treatment, prevention, or delay of a cardiovascular disease, hypercholesterolemia, hypertriglyceridemia, a metabolic disorder, inflammation, nephropathy, and/or Alzheimer's disease in a subject, said method comprising administering to the subject an effective amount of the compound of claim 1.

20. The method of treatment according to claim 19, wherein R is hydroxamic acid.
